(19) United States
(12) Patent Application Publication
(10) Pub. No.: US 2024/0418708 A1
(43) Pub. Date: Dec. 19, 2024

(54) METHODS FOR DETERMINING RESPIRATORY INFECTION RISK

(71) Applicant: Respiradigm Pty Ltd, Spearwood, Western Australia (AU)

(72) Inventors: Anthony BOSCO, Spearwood, Western Australia (AU); James READ, Spearwood, Western Australia (AU)

(21) Appl. No.: 18/704,190

(22) PCT Filed: Oct. 26, 2022
(86) PCT No.: PCT/AU2022/051283

§ 371 Date: Apr. 24, 2024

(30) Foreign Application Priority Data
Oct. 26, 2021 (AU) 2021903424

(51) Int. Cl. G01N 33/50; C12Q 1/6883

(52) U.S. Cl. CPC G01N 33/5091; C12Q 1/6883; C12Q 2600/118; C12Q 2600/158; G01N 2800/12; G01N 2800/50

(57) ABSTRACT

The present invention relates to methods for determining susceptibility to respiratory infections. In another aspect, the present invention provides a method for determining whether an individual has increased susceptibility to respiratory infections, the method comprising: contacting cord blood mononuclear cells (CBMCs) from the individual with a TLR4 agonist; measuring levels of expression of biomarkers in the CBMCs; wherein differential expression of biomarkers in the CBMCs contacted with the TLR4 agonist compared to CBMCs not contacted with the TLR4 agonist indicates the individual has increased susceptibility to respiratory infections.

### FIELD OF THE INVENTION

The present invention relates to methods for the identification of individuals who are susceptible to, are predisposed to, have a predisposition to or have an increased risk of, respiratory infections, eg severe lower respiratory tract infections, at an early stage of life. This provides an opportunity for intervention in the form of pre-emptive therapy.

### RELATED APPLICATION

This application claims priority from Australian provisional application no. 2021903424 filed 26 Oct. 2021, the entire contents of which are herein incorporated by reference in its entirety.

### BACKGROUND OF THE INVENTION

Severe lower respiratory tract infections (sLRIs) are a leading cause of emergency room presentations in infants and children, and are a major risk factor for the development of asthma and wheeze. Studies from a series of prospective birth cohorts have found that associations between sLRI and asthma are strongest in children with Rhinovirus (RV) wheezing and early aeroallergen sensitization. However, RV can routinely be detected in asthmatic children in the absence of significant symptoms, suggesting that RV may be necessary but not sufficient to drive the pathogenesis of sLRIs. In this regard it has been demonstrated that the presence of bacterial pathogens, including Moraxella, Streptococcus, and Haemophilus species, coinciding with and/or preceding viral detection can markedly amplify ensuing airway symptoms and increase risk for the subsequent development of asthma. Conversely, exposure to microbes and their products during early childhood has also been shown to protect against asthma, perhaps most elegantly described through the “farm effect”.

The underlying immunological mechanisms that determine why some individuals are more susceptible to sLRIs in early life, and subsequent asthma, are not well understood.

There remains a need to identify individuals at increased susceptibility to sLRIs, particularly individuals in early-stages of life, at a time when the immune system is not yet fully developed.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a systems biology approach to characterise innate immune responses in cord blood to a panel of stimuli (LPS, Poly(I:C), Imiquimod) across multiple layers of biological regulation (transcriptome and proteome), and identify innate immune response patterns that are associated with risk for sLRIs in the early stages of life.

In another aspect, the present invention provides a method for determining whether an individual has increased susceptibility to respiratory infections, the method comprising:
contacting B and T cells from cord blood from the individual with a TLR4 agonist;
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs;
wherein differential expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs contacted with the TLR4 agonist compared to CBMCs not contacted with the TLR4 agonist indicates the individual has increased susceptibility to respiratory infections.

In any aspect, the T cells may be CD4+ and/or CD8+ T cells. In one embodiment, the CD4+ T cells are central memory cells. In one embodiment, the CD8+ T cells are central memory cells.

In another aspect, the present invention provides a method for determining whether an individual has increased susceptibility to respiratory infections, the method comprising:
contacting cord blood mononuclear cells (CBMCs) from the individual with a TLR4 agonist;
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs;
wherein differential expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs contacted with the TLR4 agonist compared to CBMCs not contacted with the TLR4 agonist indicates the individual has increased susceptibility to respiratory infections.

In another aspect, the present invention provides a method for determining whether an individual has increased susceptibility to respiratory infections, the method comprising:
contacting B and T cells from cord blood from the individual with a TLR4 agonist;
measuring level of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of the biomarkers KLHDC7B, IFNG, CASZ1, PSMB9, PARP3, ACOT7, NUB1, USP18, NLRC5, CCDC194, GCH1, PARP11, CXCL11 and PMAIP1 in the individual compared to a reference data set indicates the individual is at increased susceptibility to respiratory infections.

In another aspect, the present invention provides a method for determining whether an individual has increased susceptibility to respiratory infections, the method comprising:
contacting cord blood mononuclear cells (CBMCs) from the individual with a TLR4 agonist;
measuring level of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of the biomarkers KLHDC7B, IFNG, CASZ1, PSMB9, PARP3, ACOT7, NUB1, USP18, NLRC5, CCDC194, GCH1, PARP11, CXCL11 and PMAIP1 in the individual compared to a reference data set indicates the individual is at increased susceptibility to respiratory infections.

In another aspect, the present invention provides a method for determining whether an individual has increased susceptibility to respiratory infections, the method comprising:
contacting B and T cells from cord blood from the individual with a TLR4 agonist;
measuring levels of expression of interferon module biomarkers;
wherein differential expression of interferon module biomarkers in the B and T cells contacted with the TLR4 agonist compared to B and T cells not contacted with the TLR4 agonist indicates the individual is at increased susceptibility to respiratory infections.

In another aspect, the present invention provides a method for determining whether an individual has increased susceptibility to respiratory infections, the method comprising:
contacting cord blood mononuclear cells (CBMCs) from the individual with a TLR4 agonist;
measuring levels of expression of interferon module biomarkers;
wherein differential expression of interferon module biomarkers in the CBMCs contacted with the TLR4 agonist compared to CBMCs not contacted with the TLR4 agonist indicates the individual is at increased susceptibility to respiratory infections.

In another aspect, the present invention provides a method for determining whether an individual has increased susceptibility to respiratory infections, the method comprising:
contacting B and T cells from cord blood from the individual with a TLR4 agonist;
measuring level of expression of interferon module biomarkers in the B and T cells;
comparing the level of expression of the biomarkers from the individual to a reference data set, wherein the reference data set comprises information on the levels of expression of the same biomarkers in B and T cells contacted with a TLR4 agonist from one of more individual individuals with or without an increased susceptibility to respiratory infections;
wherein the levels of expression of interferon module biomarkers in the individual compared to a reference data set indicates the individual is at increased susceptibility to respiratory infections.

In another aspect, the present invention provides a method for determining whether an individual has increased susceptibility to respiratory infections, the method comprising:
contacting cord blood mononuclear cells (CBMCs) from the individual with a TLR4 agonist;
measuring level of expression of interferon module biomarkers in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of interferon module biomarkers in the individual compared to a reference data set indicates the individual is at increased susceptibility to respiratory infections.

In any aspect, interferon module biomarkers are those listed in Table 3.

In another aspect, the present invention provides a method for determining whether an individual has increased susceptibility to respiratory infections, the method comprising:
contacting B and T cells from cord blood from the individual with a TLR4 agonist;
measuring levels of expression of IFN module biomarkers regulated by the IRF1 regulon;
wherein differential expression of IFN module biomarkers regulated by the IRF1 regulon in the B and T cells contacted with the TLR4 agonist compared to B and T cells not contacted with the TLR4 agonist indicates the individual is at increased susceptibility to respiratory infections.

In another aspect, the present invention provides a method for determining whether an individual has increased susceptibility to respiratory infections, the method comprising:
contacting cord blood mononuclear cells (CBMCs) from the individual with a TLR4 agonist;
measuring levels of expression of IFN module biomarkers regulated by the IRF1 regulon;
wherein differential expression of IFN module biomarkers regulated by the IRF1 regulon in the CBMCs contacted with the TLR4 agonist compared to CBMCs not contacted with the TLR4 agonist indicates the individual is at increased susceptibility to respiratory infections.

In another aspect, the present invention provides a method for determining whether an individual has increased susceptibility to respiratory infections, the method comprising:
contacting B and T cells from cord blood from the individual with a TLR4 agonist;
measuring level of expression of IFN module biomarkers regulated by the IRF1 regulon in the B and T cells;
comparing the level of expression of the biomarkers from the individual to a reference data set, wherein the reference data set comprises information on the levels of expression of the same biomarkers in B and T cells contacted with a TLR4 agonist from one of more individual individuals with or without an increased susceptibility to respiratory infections;
wherein the levels of expression of IFN module biomarkers regulated by the IRF1 regulon in the individual compared to a reference data set indicates the individual is at increased susceptibility to respiratory infections.

In another aspect, the present invention provides a method for determining whether an individual has increased susceptibility to respiratory infections, the method comprising:
contacting cord blood mononuclear cells (CBMCs) from the individual with a TLR4 agonist;
measuring level of expression of IFN module biomarkers regulated by the IRF1 regulon in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of IFN module biomarkers regulated by the IRF1 regulon in the individual compared to a reference data set indicates the individual is at increased susceptibility to respiratory infections.

In any aspect, the levels of expression of biomarkers may be absolute levels or differential levels of expression.

In any aspect, IFN module biomarkers regulated by the IRF1 regulon are those listed in Table 4.

In any aspect, the method further comprises a step of applying a machine learning algorithm, preferably a random forest analysis, to the differential expression or absolute levels of expression of biomarkers thereby indicating the individual is at high risk of susceptibility to respiratory infections.

In any aspect, the individual is less than 1 year old or equal to or less than 2 years old. In any embodiment, the individual is 1 day, 2 days, 3 days, 4 days, 5 days, 6 days, 7 days, 2 weeks, 1 month, 2 months, 3 months, 6 months, 1 year or 2 years old.

In any embodiment, the individual is at least 1 day, at least 2 days, at least 3 days, at least 4 days, at least 5 days, at least 6 days, at least 7 days, at least 2 weeks, at least 1 month, at least 2 months, at least 3 months, at least 6 months, at least 1 year old but no more than 2 years old. In any embodiment, the individual is from about 1 day to about 7 days, about 1 day to about 2 weeks, about 1 week to about 6 months, about 1 month to about 6 months, about 1 month to about 3 months about 6 months to about 1 year, about 1 month to 2 years, about 6 months to 2 years, about 1 year to 2 years old.

In any aspect, the method determines increased susceptibility to respiratory infections when the individual is about 2, about 3, about 4 or about 5 years old; or 2, 3, 4, or 5 years old. In any embodiment, the method determines increased susceptibility to respiratory infections when the individual is from about 2 years to about 5 years old, from about 2 years to about 4 years, from about 2 years to about 3 years, from about 3 years to about 5 years or from about 4 years to about 5 years.

In any aspect, the method determines increased susceptibility to respiratory infections when the individual is at least 5 years old.

In any aspect, the respiratory infections are lower respiratory tract infections. In any aspect, the respiratory infections are bacterial or viral respiratory infections. In any aspect, the respiratory infections as severe lower respiratory tract infections. In any embodiment the viral respiratory infections includes Rhinovirus and Respiratory Syncytial Virus (RSV) and the bacterial respiratory infections include Haemophilus influenzae, Staphylococcus aureus, and Moraxella spp.

In any aspect, a differential expression is a greater than 1.5-fold increase or decrease. In any embodiment, the biomarkers with a differential decrease include IF16, IF127, RHEBL1, CCDC194, BATF2, CARD16, IFIT1, ISG20, IFITM3, VAMP5, TNFSF13B, SAMD9, RNF213-AS1, IFIT2, XRN1, CD38, LRRN2 and CCDC194. In any embodiment, the biomarkers with a differential increase include biomarkers described herein, including listed in Tables 3 and 4, other than IF16, IF127, RHEBL1, CCDC194, BATF2, CARD16, IFIT1, ISG20, IFITM3, VAMP5, TNFSF13B, SAMD9, RNF213-AS1, IFIT2, XRN1, CD38, LRRN2 and CCDC194.

In another aspect, the present invention provides a method of measuring the levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in CBMCs stimulated with a TLR4 agonist.

In another aspect, the present invention provides a method of measuring the levels of expression of interferon module biomarkers in B and T cells, or CBMCs, stimulated with a TLR4 agonist.

In another aspect, the present invention provides a method of measuring the levels of expression of IFN module biomarkers regulated by the IRF1 regulon in B and T cells, or CBMCs, stimulated with a TLR4 agonist.

In another aspect, the present invention provides an assay comprising
contacting B and T cells from cord blood from the individual with a TLR4 agonist; and
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the B and T cells.

In another aspect, the present invention provides an assay comprising
contacting B and T cells from cord blood from the individual with a TLR4 agonist; and
measuring levels of expression of interferon module biomarkers in the B and T cells.

In another aspect, the present invention provides an assay comprising
contacting B and T cells from cord blood from the individual with a TLR4 agonist; and
measuring levels of expression of IFN module biomarkers regulated by the IRF1 regulon in the B and T cells.

In another aspect, the present invention provides an assay comprising
contacting cord blood mononuclear cells (CBMCs) from the individual with a TLR4 agonist; and
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs.

In another aspect, the present invention provides an assay comprising
contacting cord blood mononuclear cells (CBMCs) from the individual with a TLR4 agonist; and
measuring levels of expression of interferon module biomarkers in the CBMCs.

In another aspect, the present invention provides an assay comprising
contacting cord blood mononuclear cells (CBMCs) from the individual with a TLR4 agonist; and
measuring levels of expression of IFN module biomarkers regulated by the IRF1 regulon in the CBMCs.

In another aspect, the present invention provides a method comprising measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in CBMCs that have been contacted with a TLR4 agonist.

In another aspect, the present invention provides a method comprising measuring levels of expression of interferon module biomarkers in B and T cells, or CBMCs, that have been contacted with a TLR4 agonist.

In another aspect, the present invention provides a method comprising measuring levels of expression of IFN module biomarkers regulated by the IRF1 regulon in B and T cells, or CBMCs, that have been contacted with a TLR4 agonist.

In any aspect, the B and T cells, or CBMCs, are, or have been, contacted with the TLR4 agonist for at least 4, 6, 12, 18 or 24 hours.

In any aspect, CBMCs, are, or have been, contacted with the TLR4 agonist and then the expression level of the relevant biomarkers in B and T cells only are measured or determined.

In any aspect or embodiment, the biomarker is a protein, nucleic acid, for example RNA, or amplification product. Where the biomarker is a nucleic acid or amplification product, the method includes determining the level or amount of expression of the gene or RNA. Preferably, the biomarker is one or more nucleic acids comprising nucleotide sequences from genes or RNA transcripts of genes.

In any aspect or embodiment described herein, where there is reference to a RNA or an amplification product thereof, the invention also includes determining or measuring the presence of, level of or amount of, as the case may be, the corresponding protein (that was translated from the RNA).

In any aspect of the present invention, the level or amount of one or more biomarkers may be the level or amount of RNA. Preferably, wherein the RNA is any one of pre-mRNA or mature mRNA, and wherein changes to level or amount of RNA may be determined using any method described herein, including RNA sequencing.

In any aspect, the method further comprises administering a treatment to the individual that lowers susceptibility to respiratory infections. Preferably the treatment is palivizumab, prednisolone, omalizumab or a polybacterial formulation, or any combinational thereof.

In any aspect, the B and T cells, or CBMCs, may be purified from cord blood. Alternatively in any aspect, the B and T cells, or CBMCs, may be present in a sample of cord blood from an individual. In any embodiment, the B and T cells, or CBMCs, are present in cord blood such that any reference herein to contacting B and T cells, or CBMCs, with a TLR4 agonist includes contacting cord blood containing B and T cells, or CBMCs (for example, untreated, whole or unpurified cord blood) with a TLR4 agonist. In one embodiment, the cord blood may be depleted of erythrocytes. In one embodiment, erythrocytes are not present, or not present at significant levels, when B and T cells, or CBMCs, are contacted with a TLR4 agonist. In another embodiment, erythrocytes are present at normal levels, i.e. they have not been depleted from the cord blood.

In any aspect, the CBMCs comprise or consist of lymphocytes (T and B cells). Preferably, the CBMCs further comprise CD14+ monocytes and conventional dendritic cells (cDCs) and optionally plasmacytoid DCs (pDC). In any embodiment, the CBMCs comprise or consist of CD4+ T cells, CD8+ T cells, and B cells.

In any aspect, the TLR4 agonist is any one described herein. Preferably, the TLR4 agonist is derived from a bacterium. More preferably, the TLR4 agonist is LPS. In any embodiment, the LPS may be purified. Alternatively, the LPS may be contained in a bacterial preparation.

In another aspect, the invention provides a kit, panel or microarray comprising at least two diagnostic reagents described herein, each reagent identifying a different biomarker described herein. In one embodiment, the kit comprises diagnostic reagents that bind to or complex individually with 2, 3, 4, 5, 6, 7, 8, 9, 10, 11, 12, 13, 14 or more biomarkers. In one embodiment, the kit may comprise diagnostic reagents that bind to or complex individually with each of the biomarkers:
KLHDC7B, IFNG, CASZ1, PSMB9, PARP3, ACOT7, NUB1, USP18, NLRC5, CCDC194, GCH1, PARP11, CXCL11 and PMAIP1;
interferon module biomarkers listed in Table 3; or
IFN module biomarkers regulated by the IRF1 regulon listed in Table 4.

In another aspect, there is provided a kit for use or when used according to a method of the present invention comprising at least two diagnostic reagents described herein, each reagent identifying a different biomarker described herein. In one embodiment, the kit comprises diagnostic reagents that bind to or complex individually with 2, 3, 4, 5, 6, 7, 8, 9, 10, 11, 12, 13, 14 or more biomarkers. In one embodiment, the kit may comprise diagnostic reagents that bind to or complex individually with each of the biomarkers:
KLHDC7B, IFNG, CASZ1, PSMB9, PARP3, ACOT7, NUB1, USP18, NLRC5, CCDC194, GCH1, PARP11, CXCL11 and PMAIP1;
interferon module biomarkers listed in Table 3; or
IFN module biomarkers regulated by the IRF1 regulon listed in Table 4.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Dimensionality reduction of multi-omics datasets. (A) Schematic representation of experimental and analysis design. (B) Immunophenotyping of baseline CBMC samples. Y-axis shows cell type proportion of total cell types identified from CBMC. Scatterplot shows median with 95% CI. (C) Multi-level dimensionality reduction for gene expression (PCA), cytokine (PCA) datasets. Axis show proportion of the total variation (%) accounted for by the first (x) and second (y) principal components or canonical variates. (D-E) Vertical bar plots showing top contributing features for the first (top row) and second (bottom row) principal components or cross validated canonical variates for the corresponding (above) dimensionality reduction plots. x-axis shows absolute contribution (%)/loading; red indicates positive/higher and blue indicates negative/lower.

FIG. 2. Differential expression and network analysis identified interferon and proinflammatory gene expression characterise innate CBMC responses. (A-C; left panel) Volcano plot showing significantly upregulated (right; red) and downregulated (left; blue) gene compared to matched unstimulated samples for the LPS, Imiquimod, and Poly(I:C) responses, respectively. Arrows indicate the number of upregulated and downregulated genes. (A-C; right panel) Pathways overrepresented from significantly upregulated genes of the CBMC LPS, Imiquimod, and Poly(I:C) responses compared to matched unstimulated controls, respectively. (D-F) Modules identified from network analysis (WGCNA) of the LPS, Imiquimod, and Poly(I:C) responses, respectively. Groups are in the order of the figure key as shown top to bottom. Modules are plotted by moderated t-statistic (limma/voom) and show the median, 25th and 75th quartiles, ±1.5×IQR, and outliers. Modules with medians above the red line (top line) (moderated t-statistic=2) are considered significantly upregulated and those below the blue line (bottom line) (−2) are considered significantly downregulated. Modules are labelled left to right in the same order of the legend. (G) Bar plot of the number of genes in the interferon and proinflammatory modules for the respective responses. (H) Heatmap showing Spearman's Rho values of ranked expression and connectivity between CBMC response module genes. Expression of member genes from the interferon and proinflammatory modules of each response was correlated against the expression of the same genes from the other responses. The p value associated with all correlations was <0.01.

FIG. 3. IFN module gene connectivity and drivers of CBMC responses, and between birth 5 years of age. (A) Density plot of the LPS, Imiquimod, and Poly(I:C) CBMC response IFN module connectivity, respectively. Dashed lines denote mean (light grey) and median (dark grey). Lillefors p value >0.05 indicate normally distributed connectivity. (B-D; left panel) Network wiring diagrams of the top 20 most connected genes for the LPS, Imiquimod, and Poly(I:C) CBMC IFN modules, respectively. Node size represents number of connections (degree) among the total network and edge with indicates strength of connection (red edges, ie darker lines, denote a correlation >0.8). (B-D; right panel) Top 10 master regulators identified by VIPER analysis for the LPS, Imiquimod, and Poly(I:C) CBMC IFN modules. Bar plots show normalized enrichment score (NES) for transcription factors which are significantly activate (NES>2, red line) or inactive/inhibited (NES<−2, blue line). Grey shading indicates an adjusted P value <0.05. (E) Network wiring diagram of the top 20 most connected cord blood LPS-induced IFN module genes from matched CBMC (i) and 5 year PBMC (ii) samples. Network characteristics are the same as above (FIG. 3B-D). (F) Network connectivity density plot of the cord blood interferon module gene connectivity of the matched CBMC and 5 year PBMC responses to LPS stimulation. (G) Top significant drivers of the cord blood interferon module genes identified for matched CBMC (i) and 5 year PBMC (ii, n=9 significant drivers) samples. Bar plot characteristics are the same as above (FIG. 3C). (H&I) Network connectivity density plot of the cord blood interferon module gene connectivity of the matched CBMC and 5 year PBMC responses to Imiquimod and Poly(I:C) stimulation, respectively.

FIG. 4. LPS-induced IFN genes predict sLRI susceptibility at birth. (A) Random forest classifiers were trained on LPS-, Imiquimod, and Poly(I:C)-induced IFN module genes from 25 (50%) randomly selected study subjects and validated on the remaining 25 (50%) subjects. Each RF model was optimised with respect to the number of genes used at each split and number of trees grown. Plot depicts the area under the Receiver Operator Characteristic (ROC) curve defined by the rate of false (x-axis, 1-specificity) and true (y-axis, sensitivity) positives. (B) RF model predictions were repeated by re-sampling the training/validation set (50/50 random assignment) 2,000 times. Plot show the area under the ROC curve for each re-sample, with median (solid lines) and 95% CIs (dashed lines). (C) Network connectivity density plot of the LPS- (i), Imiquimod- (ii) and Poly(I:C)- (iii) induce IFN module gene network stratified by individuals who did (orange; ie lighter line) and did not (grey) record an sLRI in the first year of life. (D-E (i)) Network wiring diagram of the top 20 most connected genes of the LPS-induced IFN module gene network from CBMC samples from individuals who were resistant (D(i)) and susceptible (E(i)) to sLRIs in infancy. Node and edge characteristics are the same as FIG. 3B. (D-E (ii)) Top 10 master regulators identified by VIPER analysis for the CBMC LPS-induced IFN response module for individuals who were resistant (D(ii)) and susceptible (E(ii)) to sLRIs in infancy. Bar plot characteristics are the same FIG. 3C. (F) Box and whisker plot of the cord blood LPS-induced IFN module eigengene, grouped by individuals who did (orange; ie lighter line) and did not (grey) record an sLRI in the first year of life. Boxes show median, 25th and 75th quartiles, 1.5×IQR, and outliers; P value determined by Mann-Whitney U test. (G) Plot of IFN module eigengenes from left to right: LPS (green; groups 1, 2, 7 and 8)), Imiquimod (blue; groups 3, 4, 9 and 10) and Poly(I:C) (red; groups 5, 6, 11 and 12) CBMC responses grouped by individuals who were resistant (−) and susceptible (+) to LRIs and sLRIs in infancy. P values determined by Mann-Whitney U test and significant result reflects FIG. 4G. Plot shows median (symbol) and 95% CI (bars).

FIG. 5. Validation of in vitro CBMC culture IFN module genes in external gene expression datasets, multi-omic integration of LPS-induced biological features, and IRF1 gene expression correlations. (A-C) A random forest classifier was trained on Unstimulated and LPS or Imiquimod/Poly(I:C) CBMC gene expression data (n=50) and used to predict; (A) children (<17 yrs) hospitalized with bacterial (n=52) and viral infections (n=92), respectively, from healthy controls (n=52) from blood-derived gene expression profiles, (B) infants (<18 mo; n=15) and children (18 mo-5 yrs; n=16) presenting to hospital with acute viral respiratory infections compared to convalescence from PBMC samples; and (C) asthmatic children (6-17 yrs) with cold-like symptoms who do (n=193) and do not (n=105) have detectable airway viral infection from nasal-derived gene expression profiles. Plots depict the area under the ROC curve. (D) Multi-layer risk profile for sLRI susceptibility in infancy determined from multi-omic data integration. Between layer co-expression was maximised and positive (red) and negative (blue) correlations stronger that ±0.8 are shown, respectively. Peripheral lines represent the relative expression of features from individuals who were resistant (grey) or susceptible (orange; ie lighter line) to sLRIs in the first year of life. Input data was adjusted with respect to matched unstimulated samples (except baseline immunophenotype data). “R_” was added to transcription factor IDs (green) to differentiate from gene names (blue). (E-G) Plot of the association between LPS-induced IRF1 gene expression with IFN and proinflammatory genes (G), viral-related receptors (H), and chemo/cytokines (1). Data was adjusted with respect to matched unstimulated samples and plots shows Spearman's Rho value (symbol) and 95% CI (bars, 1000 bootstraps); Red and blue data points/labels denote positive and negative correlations (ie non-overlapping 95% confidence intervals greater than or less than 0, respectively), with a BH-adjusted FDR<0.05. It will be understood that the invention disclosed and defined in this specification extends to all alternative combinations of two or more of the individual features mentioned or evident from the text or drawings. All of these different combinations constitute various alternative aspects of the invention.

FIG. 6. (A) Ten selected significantly overrepresented pathways (InnateDB) for genes contained within the IFN module of the CBMC responses to the LPS. (B) Original VIPER plot (before trimming by motif binding sites) of the LPS CBMC response interferon module. The plots show (L-R) p value, positive (right; red) and negative (left; blue) interactions, transcription factor gene name, activated (top panels; red) or inactivated/inhibited (bottom panels; blue) status (NES), and relative expression of the TF genes.

FIG. 7. Density plot and putative driver analysis (as previously described) of cord blood CBMC responses when the input genes are restricted to only those genes common to the LPS-induced IFN module.

FIG. 8. Drivers for the CBMC Imiquimod (A) and Poly(I:C) (B) induced IFN modules for the matched CBMC (left) and 5 yr PBMC (right) samples.

FIG. 9. Top 30 genes most important genes for the CBMC LPS-(A), Imiquimod- (B), and Poly(I:C)-induced (C) IFN module random forest classifiers, respectively. The x-axis indicates the accuracy loss for each model by excluding each variable.

FIG. 10. (A,B) RF model predictions were repeated by re-sampling the training/validation set (50/50 random assignment, 2000 re-samples) with the original (optimised) RF parameters for each model remaining consistent. The AUC-ROC for each re-sample as a density (A). Solid lines denote the median AUC-ROC, and dashed lines denote the high and low 95% confidence intervals. These values were as follows; LPS: median=0.619, CI^{lo}=0.615, CI^{hi}=0.623; Imiquimod: median=0.338, CI^{lo}=0.335, CI^{hi}=0.341; Poly(I:C): median=0.393, CI^{lo}=0.389, CI^{hi}=0.397. Dashed grey line indicates an AUC-ROC of 0.5 (random chance). (B) Re-sampled AUC-ROC values are displayed as a box-and-whisker plot. Matched re-sample permutations are connected by grey lines. The matched re-sample AUC-ROC values were significantly different between all groups (Wilcoxon SRT p value <0.00001).

FIG. 11. (A) CBMC LPS-induced IFN module differential connectivity confirmed by assessment with a separate connectivity measure (Spearman's Rho) between individuals who were resistant and susceptible to sLRIs in infancy. (B,C) Network connectivity density plots of the Imiquimod-induce (B) and Poly(I:C)-induced (C) IFN module gene networks after restricting input genes to only those common to the LPS-induced IFN module, stratified by individuals who did (orange; ie lighter line) and did not (grey) record an sLRI in the first year of life.

FIG. 12. (A) Box and whisker plot of the cord blood LPS-induced IFN module eigengene, grouped by individuals who are asthmatic/non-asthmatics at 5 years of age. (B) Box and whisker plot of the cord blood LPS-induced IFN module eigengene, grouped by individuals who did or did not have wheeze in the 5^{th} year of life.

FIG. 13. (A) Principal component analysis of blood-derived gene expression profiles of children hospitalised with febrile bacterial and viral infections (GSE72809). Gene expression data sets were restricted to available CBMC LPS-induced, Imiquimod-induced, and Poly(I:C)-induced IFN module genes, respectively. (B) Top 30 genes most important genes for the CBMC LPS-, Imiquimod-, and Poly(I:C)-induced IFN module random forest classifiers, respectively.

FIG. 14. (A-C) Analysis of IFN and proinflammatory mediators and viral-related receptor genes with respect to sLRI susceptibility in the first year of life. Data was adjusted with respect to matched unstimulated samples and plots show the Mann-Whitney U test estimates and 95% CIs for CBMC data of individuals who are susceptible compared to resistant to sLRIs in infancy. Red data points/labels (darker labels) indicate increase expression with a p value <0.05. (D) Spearman's correlation and associated p value between IFIH1 and IRF1/STAT1 among CBMC (n=50) stimulated with LPS. Dashed blue line represents loess fit of the data.

FIG. 15. Differential gene expression from single cell RNA sequencing analysis comparing lymphoid cells collected from cord blood with or without LPS treatment. Genes coloured red (darker points located on the right side of the rightmost dotted line) are considered upregulated and genes coloured blue (darker points located on the left side of the leftmost dotted line) are considered downregulated.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The invention is based on the development of a method that can predict at birth which children will experience respiratory infections in early life, including those viral infections that are associated with the subsequent development of asthma. The present inventors' findings suggest that susceptibility to severe respiratory viral infections (e.g. sLRI) in the first year of life is primarily determined by anti-bacterial versus anti-viral innate immune pathways, and provides a rationale for identification of at-risk infants for early intervention. In this regard, the data presented herein suggests that responses to pathogenic bacteria are more important determinants of sLRI susceptibility than responses to viral stimuli.

The results described herein of heightened bacterial mediated TLR4-induced IFN responses/gene network connectivity patterns at birth conferred risk of viral sLRIs in infancy is surprising given that IFN responses are almost universally protective during acute viral infections

Every year, thousands of children present to the emergency departments with severe respiratory viral infections. Severe viral lower respiratory tract infections (sLRIs) are a leading cause of hospitalization in infants and children and constitute a major risk factor for subsequent asthma development.

Moreover, a subset of these children will develop asthma, which is a chronic inflammatory disease of the airways that affects 300 million people worldwide. Notably, the trajectory towards asthma beings in utero and during the first few years of life, which represents a crucial period of heightened plasticity where the immune system is functionally immature and highly susceptible to infection. The plasticity of the immune system in early infancy provides an ideal “window of opportunity” for administration of immunomodulatory drugs to reprogram the immune system and minimise disease risk. The present invention enables the very early identification of high-risk infants, which can be treated with appropriate interventions to modulate innate immunity and reduce or prevent the development of severe respiratory viral infections and subsequent asthma.

Accordingly, the early identification of high-risk infants enables these children to be treated with immunomodulatory drugs, which in turn will minimise risk for the development of severe respiratory viral infections and subsequent asthma. Prevention of emergency room presentations due to severe respiratory viral infections will save the health care system billions of dollars and improve the quality of life for millions of children and their families.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

As used herein, a TLR4 agonist may be selected from the group consisting of lipopolysaccharide (LPS), monophosphoryl lipid A (MPLA), a heat shock protein, S100A8, S100A9, RSV F protein, fibrinogen, heparin sulfate or a fragment thereof, hyaluronic acid or a fragment thereof, nickel, an opoid, α1-acid glycoprotein (AAG), aminoakyl glucoaminide 4-phosphate (AGP), RC-529, murine β-defensin 2, and complete Freund's adjuvant (CFA).

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular biomarkers, including those described herein, may be characteristic of increased susceptibility (i.e., increased risk) of respiratory infection (e.g. sLRI), for example as characterized by a relative risk (RR) or odds ratio (OR) of greater than one for the particular biomarkers. Alternatively, the biomarkers are characteristic of decreased susceptibility (i.e., decreased risk) of respiratory infection (e.g. sLRI), as characterized by a relative risk of less than one.

Measures of susceptibility or risk include measures such as relative risk (RR), odds ratio (OR), and absolute risk (AR), as described in more detail herein.

In certain embodiments, increased susceptibility refers to a risk with values of RR or OR of at least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.30, at least 1.35, at least 1.40, at least 1.45, at least 1.50, at least 1.55, at least 1.60, at least 1.65, at least 1.70, at least 1.75, and/or at least 1.80. Other numerical non-integer values greater than unity are also possible to characterize the risk, and such numerical values are also within scope of the invention.

Increased susceptibility may also involve comparison with a reference data set. A reference data set may be from one or more individuals who (a) have been determined to be at an increased, elevated, high or higher risk or susceptibility of respiratory infections (e.g. sLRI) (also referred to as increased or high risk reference data set) or (b) have been determined to be at no-increased risk or susceptibility of respiratory infections (also referred to as the normal or no increased risk reference data set). Therefore, if the levels of expression of biomarkers in the sample from the individual in whom the risk is to be determined is the same or not significantly different to the increased or high risk reference data set, then a determination may be made that the individual does have an increased or high risk of respiratory infections. Alternatively, if the levels of expression of biomarkers in the sample from the individual in whom the risk is to be determined is significantly different to the increased or high risk reference data set, then a determination may be made that the individual does not have an increased or high risk of respiratory infections. Alternatively, if the levels of expression of biomarkers in the sample from the individual in whom the risk is to be determined is the same or not significantly different to the normal or no increased risk reference data set, then a determination may be made that the individual does not have an increased or high risk of respiratory infections. Alternatively, if the levels of expression of biomarkers in the sample from the individual in whom the risk is to be determined is significantly different to the normal or no increased risk reference data set, then a determination may be made that the individual does have an increased or high risk of respiratory infections.

In any method or use of the invention, the determination may be increased, elevated, high or higher risk or susceptibility of respiratory infection (e.g. sLRI), or the determination may be no increased, no elevated, no higher or normal risk or susceptibility of respiratory infection. As used herein, reference to a determination of increased, elevated, high or higher risk or susceptibility of respiratory infection may be taken as a reference to determination that an individual requires an intervention in the form of pre-emptive therapy. Therefore, in any method or use of the invention, where a determination is made that an individual requires an intervention in the form of pre-emptive therapy, the method or use further comprises the step of administering an intervention in the form of pre-emptive therapy (for example, any pre-emptive therapy described herein).

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The term “microarray” refers to an ordered arrangement of binding/complexing array elements or ligands, e.g. antibodies, on a substrate.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, the term “subject” shall be taken to mean any animal including humans, for example a mammal. Exemplary subjects include but are not limited to humans and non-human primates. For example, the subject is a human.

### Samples

In any aspect, the B and T cells, or CBMCs, may be purified from cord blood. Alternatively in any aspect, the B and T cells, or CBMCs, may be present in a sample of cord blood from an individual. In any embodiment, the B and T cells, or CBMCs, are present in cord blood such that any reference herein to contacting CBMCs with a TLR4 agonist includes contacting cord blood containing the B and T cells, or CBMCs, (e.g. untreated, whole or non-purified cord blood) with a TLR4 agonist. In one embodiment, the cord blood may be depleted of erythrocytes. In one embodiment, erythrocytes are not present, or not present at significant levels, when B and T cells, or CBMCs, are contacted with a TLR4 agonist. In another embodiment, erythrocytes are present at normal levels, i.e. they have not been depleted from the cord blood.

In any aspect, the individual is less than 1 year old or equal to or less than 2 years old. In any embodiment, the individual is 1 day, 2 days, 3 days, 4 days, 5 days, 6 days, 7 days, 2 weeks, 1 month, 6 months, 1 year or 2 years old. In any embodiment, the individual is at least 1 day, at least 2 days, at least 3 days, at least 4 days, at least 5 days, at least 6 days, at least 7 days, at least 2 weeks, at least 1 month, at least 2 months, at least 3 months, at least 6 months, at least 1 year but no more than 2 years old. In any embodiment, the individual is from about 1 day to about 7 days, about 1 day to about 2 weeks, about 1 week to about 6 months, about 1 month to about 6 months, about 1 month to about 3 months or about 6 months to about 1 year, about 1 month to 2 years, about 6 months to 2 years, about 1 year to 2 years old.

In any embodiment, the cord blood is derived from an individual within 24 hours of birth. In any embodiment, the cord blood is derived from an individual within 48 hours. The cord blood may be obtained and then frozen for up to 2 years prior to use in the methods of the present invention. For example, the cord blood may be obtained and frozen for an amount of time less or equal to the current age of the individual whom is subject to the methods of the present invention as described herein. Accordingly, the CBMCs may be obtained from fresh cord blood within 24 hours or within 48 hours or cord blood that has been frozen for up to 2 years from birth of an individual.

In any embodiment, cord blood erythrocytes are depleted by ammonium chloride lysis, density gradient technique, hypotonic lysis, immunomagnetic cell separation or sedimentation, flow cytometric sorting, or equivalent methods appreciated by those skilled in the art.

In any embodiment, CBMCs are cultured in a nutrient medium at or near physiological conditions. For example CBMCs may be cultured in RPMI+5% AB non-heat inactivated serum at 37° C., 5% CO₂. In any embodiment, CBMCs are cultured in a nutrient medium containing non-heat inactivated serum.

In any embodiment, the B and T cells cultured in a nutrient medium at or near physiological conditions. B and T cell cultured medium are known in the art.

Stimulation with TLR4 Agonists

In any aspect of the invention, the B and T cells, or CBMCs, or cord blood may be stimulated with a TLR4 agonist.

B and T cells, or CBMCs, or cord blood may be stimulated with a TLR4 agonist for at least 6 hours, at least 12 hours, at least 18 hours or at least 24 hours. In any embodiment, CBMCs may be suspended at 1×10⁶ cells/mL and then stimulated with a TLR4 agonist.

In any embodiment, the TLR4 agonist is provided at an effective concentration to stimulate TLR4 activation. An example of an effective concentrate is between 0.025 ng/ml to 100 ng/ml, preferably 1 ng/ml, of LPS. For other TLR4 agonists, the effective concentration is any amount that provides the same activation of TLR4 as 1 ng/ml of LPS. One skilled in the art, as described herein, would also recognise the methods that can be used to determine an effective concentration of any TLR4 agonist to stimulate TLR4 activation. For example the skilled person may perform an assay to the effective concentration of any TLR4 agonist. An exemplary assay includes stimulating B and T cells, CBMCs or cord blood in vitro with a TLR4 agonist overnight and measuring NK-κB mediated transcriptional activity compared to unstimulated B and T cells, CBMCs, or cord blood, respectively, wherein an increase in NK-κB mediated transcriptional activity is indicative of an effective concentration of TLR4 agonist.

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules that are indicative of the expression level of the biomarker.

In one embodiment, the expression levels of the biomarkers are determined by measuring polynucleotide levels of the biomarkers. The levels of transcripts of specific biomarker genes can be determined from the amount of mRNA, or polynucleotides derived therefrom, present in a sample. Polynucleotides can be detected and quantitated by a variety of methods including, but not limited to, microarray analysis, polymerase chain reaction (PCR), reverse transcriptase polymerase chain reaction (RT-PCR), Northern blot, serial analysis of gene expression (SAGE), total RNA-Sequencing, mRNA-Sequencing, Cap analysis gene expression (CAGE) sequencing, single cell RNA-Sequencing, or NanoString nCounter. See, e.g., Draghici Data Analysis Tools for DNA Microarrays, Chapman and Hall/CRC, 2003; Simon et al. Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; herein incorporated by reference in their entireties.

In one embodiment, microarrays are used to measure the levels of biomarkers. An advantage of microarray analysis is that the expression of each of the biomarkers can be measured simultaneously, and microarrays can be specifically designed to provide an expression profile for a particular disease or condition, regulon or network.

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm² and 25 cm²; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, or to a specific cDNA derived therefrom).

However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one embodiment, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphor amidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93: 10539-11286 (1995); herein incorporated by reference in their entireties).

A second method for making microarrays produces high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14: 1675; U.S. Pat. Nos. 5,578,832; 5,556,752; and 5,510,270; herein incorporated by reference in their entireties) or other methods for rapid synthesis and deposition of defined oligonucleotides (Blanchard et al., Biosensors & Bioelectronics 11:687-690; herein incorporated by reference in its entirety). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20: 1679-1684; herein incorporated by reference in its entirety), may also be used. In principle, any type of array, for example, dot blots on a nylon hybridization membrane (see Sambrook, et al., Molecular Cloning: A Laboratory Manual, 3rd Edition, 2001) could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123; herein incorporated by reference in their entireties. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm. The polynucleotide probes are attached to the support covalently at either the 3′ or the 5′ end of the polynucleotide. Biomarker polynucleotides which may be measured by microarray analysis can be expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)+ messenger RNA (mRNA) or a fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. RNA can be fragmented by methods known in the art, e.g., by incubation with ZnCl₂, to generate fragments of RNA.

In one embodiment, total RNA, mRNA, or nucleic acids derived therefrom, are isolated from a stimulated sample. Biomarker polynucleotides that are poorly expressed in particular cells may be enriched using normalization techniques (Bonaldo et al., 1996, Genome Res. 6:791-806).

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative.

Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one embodiment, biomarker polynucleotide molecules from a sample are labelled differentially from the corresponding polynucleotide molecules of a reference sample. The reference can comprise polynucleotide molecules from a normal biological sample (i.e., control sample, e.g., stimulated CMBCs from an individual who is not susceptible to sLRI) or from a reference biological sample, (e.g., stimulated CMBCs from an individual who is susceptible to sLRI).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001), and in Ausubel et al., Current Protocols In Molecular Biology, vol. 2, Current Protocols Publishing, New York (1994). Typical hybridization conditions for the cDNA microarrays of Schena et al. are hybridization in 5.times.SSC plus 0.2% SDS at 65° C. for four hours, followed by washes at 25° C. in low stringency wash buffer (IxSSC plus 0.2% SDS), followed by 10 minutes at 25° C. in higher stringency wash buffer (O.IxSSC plus 0.2% SDS) (Schena et al., Proc. Natl. Acad. Sci. U.S.A. 93: 10614 (1993)). Useful hybridization conditions are also provided in, e.g., Tijessen, 1993, Hybridization With Nucleic Acid Probes, Elsevier Science Publishers B.V.; and Kricka, 1992, Nonisotopic Dna Probe Techniques, Academic Press, San Diego, Calif. Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labelled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14: 1681-1684 (1996), may be used to monitor mRNA abundance levels at a large number of sites simultaneously.

Polynucleotides can also be analyzed by other methods including, but not limited to, northern blotting, nuclease protection assays, RNA fingerprinting, polymerase chain reaction, ligase chain reaction, Qbeta replicase, isothermal amplification method, strand displacement amplification, transcription based amplification systems, nuclease protection (S 1 nuclease or RNAse protection assays), SAGE as well as methods disclosed in International Publication Nos. WO 88/10315 and WO 89/06700, and International Applications Nos. PCT/US 87/00880 and PCT/US89/01025; herein incorporated by reference in their entireties.

A standard Northern blot assay can be used to ascertain an RNA transcript size, identify alternatively spliced RNA transcripts, and the relative amounts of mRNA in a sample, in accordance with conventional Northern hybridization techniques known to those persons of ordinary skill in the art. In Northern blots, RNA samples are first separated by size by electrophoresis in an agarose gel under denaturing conditions. The RNA is then transferred to a membrane, cross-linked, and hybridized with a labelled probe. Nonisotopic or high specific activity radiolabeled probes can be used, including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabeled cDNA, either containing the full-length, single stranded DNA or a fragment of that DNA sequence may be at least 20, at least 30, at least 50, or at least 100 consecutive nucleotides in length. The probe can be labelled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labelled with a radioactive element or with an enzyme. The radioactive label can be detected by any of the currently available counting procedures. Isotopes that can be used include, but are not limited to, ³H, ¹⁴C, ³²P, ³⁵S, ³⁶C1, ³⁵Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluoro spectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays (including both ribonuclease protection assays and S 1 nuclease assays) can be used to detect and quantitate specific mRNAs. In nuclease protection assays, an antisense probe (labelled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 pg maximum of blot hybridizations. The ribonuclease protection assay, which is the most common type of nuclease protection assay, requires the use of RNA probes. Oligonucleotides and other single-stranded DNA probes can only be used in assays containing S 1 nuclease. The single-stranded, antisense probe must typically be completely homologous to target RNA to prevent cleavage of the probe:target hybrid by nuclease.

Serial Analysis Gene Expression (SAGE) can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, and is one of the preferable analytical methods for simultaneously detecting the expression of a large number of transcription products. First, poly A⁺ RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labelled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A more recent variation of the RT-PCR technique is the real time quantitative PCR, which measures PCR product accumulation through a dual-labelled fluorogenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Next Generation Sequencing methods such as RNA-sequencing (RNA-seq) can also be used to evaluate RNA abundance in sample cells. Exemplary protocols of performing RNA-seq include RNA ACCESS® protocol or TRUSEQ® RIBO-ZERO® protocol (ILLUMINA®). One skilled in the art would also recognise the many methods of performing RNA-seq including, but not limited to, total RNA sequencing, mRNA sequencing, 3′ mRNA sequencing, 5′ mRNA sequencing, CAGE-Seq.

Biomarker data may be analyzed by a variety of methods to identify biomarkers and determine the statistical significance of differences in observed levels of biomarkers between test and reference expression profiles in order to evaluate whether a patient is susceptible to sLRI. In certain embodiments, patient data is analyzed by one or more methods including, but not limited to, multivariate linear discriminant analysis (LDA), receiver operating characteristic (ROC) analysis, principal component analysis (PCA), ensemble data mining methods, bayesian generalized linear model, gaussian process, naive bayes, elastic net, k-nearest neighbors, lasso, penalized logistic regression, partial least squares, prediction analysis for microarrays (PAM), poisson linear discriminant analysis, negative-Binomial linear discriminant analysis, neural networks, support vector machines, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, NY: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, CA, USA; English et al. (2009) J. Biomed. Inform. 42(2):287-295; Zhang (2007) Bioinformatics 8: 230; Shen-Orr et al. (2010) Journal of Immunology 184: 144-130; Qiu et al. (2011) Nat. Biotechnol. 29(10):886-891; Ru et al. (2006) J. Chromatogr. A. 1111(2): 166-174, Jolliffe Principal Component Analysis (Springer Series in Statistics, 2^{nd} edition, Springer, NY, 2002), Koren et al. (2004) IEEE Trans Vis Comput Graph 10:459-470; herein incorporated by reference in their entireties).

A preferred method by which the biomarker data, i.e. gene expression data, is analysed is by a Random Forrest classifier.

### Random Forest Classifier

Random Forest classifiers are an ensemble classifier that consists of many decision trees and outputs the class that is the mode of the classes output by individual trees. Random Forests utilize bootstrapping instead of cross-validation. For each iteration, a random sample (with replacement) is drawn and the largest tree possible is grown. Each tree receives a vote in the final class prediction. To fit a random forest, the number of trees (e.g. bootstrap iterations) is specified. The random forest algorithm gauges biomarker importance by the average reduction in the training accuracy. The random forest method uses a number of different decision trees. A biomarker is considered to have discriminating significance if it served as a decision branch of a decision tree from a significant random forest analysis.

Random forest (or random forests) is an ensemble classifier that consists of many decision trees and outputs the class that is the mode of the classes output by individual trees. (Breiman, Leo (2001). “Random Forests”. Machine Learning 45 (1): 5-32). Random forest is one of the most accurate learning algorithms available, i.e., produces a highly accurate classifier for data sets. (Caruana, Rich; Karampatziakis, Nikos; Yessenalina, Ainur (2008). “An empirical evaluation of supervised learning in high dimensions.” Proceedings of the 25th International Conference on Machine Learning (ICML)). The method combines “bagging” and the random selection of features in order to construct a collection of decision trees with controlled variation. The selection of a random subset of features is an example of the random subspace method, which is a way to implement stochastic discrimination. Bootstrap distribution is used as a way to estimate the variation in a statistics based on the original data. For each tree grown on a bootstrap sample, e.g., 150 or 500, the error rate for observations left out of the bootstrap sample is monitored. This is called the “out-of-bag” error rate.

Each tree is constructed using the following algorithm: (1) Let the number of training cases be N, and the number of variables in the classifier be M; (2) The number m of input variables to be used to determine the decision at a node of the tree; m should be much less than M; (3) Choose a training set for this tree by choosing n times with replacement from all N available training cases (i.e., take a bootstrap sample), and use the rest of the cases to estimate the error of the tree, by predicting their classes; (4) For each node of the tree, randomly choose m variables on which to base the decision at that node. Calculate the best split based on these m variables in the training set; and (5) Each tree is fully grown and not pruned (as may be done in constructing a normal tree classifier).

For prediction a new sample is pushed down the tree. It is assigned the label of the training sample in the terminal node it ends up in. This procedure is iterated over all trees in the ensemble, and the mode vote of all trees is reported as random forest prediction.

In one embodiment, random forest analysis involving classification and regression based on a forest of trees using random inputs is performed using “randomForest: Breiman and Cutler's random forests for classification and regression” (Depends: R (>=2.5.0), stats) (Version: 4.6-6) (2012-01-06) (Fortran original by Leo Breiman and Adele Cutler, R port by Andy Liaw and Matthew Wiener). See, A. Liaw and M. Wiener (2002). Classification and Regression by randomForest. R News 2(3), 18-22.

Random Forests are further described in Liaw and Wiener, R News Vol. 2/3, December 2002, pgs. 18-22; Dfaz-Uriarte and Alvarez, BMC Bioinformatics. 2006 Jan. 6; 7:3); Statnikov et al., BMC Bioinformatics. 2008 Jul. 22; 9:319; Shi et al., Mod Pathol. 2005 April; 18(4):547-57, Breiman, 1999, “Random Forests—Random Features,” Technical Report 567, Statistics Department, U.C. Berkeley, September 1999, which is hereby incorporated by reference in its entirety, each of which is incorporated by reference herein it its entirety.

### Respiratory Infections

The present invention provides methods for determining susceptibility to respiratory infections. Preferably, the respiratory infections are lower respiratory tract infections. In any embodiment, the infections may be bacterial or viral infections. The bacterial infections may be any as described herein. The viral infections may be any as described herein.

As used herein, the term respiratory infection means an infection by virus or bacteria anywhere in the respiratory tract. Examples of respiratory infection include but are not limited to colds, sinusitis, throat infection, tonsillitis, laryngitis, bronchitis, pneumonia or bronchiolitis. Preferably, in any embodiment of the invention the respiratory infection is a cold.

An individual may be identified as having a respiratory tract infection by viral testing and may exhibit symptoms of itchy watery eyes, nasal discharge, nasal congestion, sneezing, sore throat, cough, headache, fever, malaise, fatigue and weakness. In one aspect, a subject having a respiratory infection may not have any other respiratory condition. Detection of the presence or amount of virus may be by PCR/sequencing of RNA isolated from clinical samples (nasal wash, sputum, BAL) or serology.

Influenza (commonly referred to as “the flu”) is an infectious disease caused by RNA viruses of the family Orthomyxoviridae (the influenza viruses) that affects birds and mammals. The most common symptoms of the disease are chills, fever, sore throat, muscle pains, severe headache, coughing, weakness/fatigue and general discomfort.

The influenza viruses make up three of the five genera of the family Orthomyxoviridae. Influenza Type A and Type B viruses co-circulate during seasonal epidemics and can cause severe influenza infection. Influenza Type C virus infection is less common but can be severe and cause local epidemics.

Influenza Type A virus can be subdivided into different serotypes or subtypes based on the antibody response to these viruses. Influenza A viruses are divided into subtypes based on two proteins on the surface of the virus: the hemagglutinin (H) and the neuraminidase (N). There are 18 different hemagglutinin subtypes and 11 different neuraminidase subtypes. (H1 through H18 and N1 through N11 respectively.) The sub types that have been confirmed in humans are H1N1, H1N2, H2N2, H3N2, H5N1, H7N2, H7N3, H7N7, H9N2 and H10N7.

Influenza has an enormous impact on public health with severe economic implications in addition to the devastating health problems, including morbidity and even mortality. Accordingly, there is a need for therapeutic agents which can prevent infection, or reduce severity of infection in individuals.

In any embodiment, the influenza infection for which prevention is required is an infection with a virus selected from the group consisting of influenza Types A, B or C. Influenza Type A virus can be subdivided into different serotypes or subtypes based on the antibody response to these viruses. Influenza A viruses are divided into subtypes based on two proteins on the surface of the virus: the hemagglutinin (H) and the neuraminidase (N). There are 18 different hemagglutinin subtypes and 11 different neuraminidase subtypes (H1 through H18 and N1 through N11 respectively). The sub types that have been confirmed in humans are H1N1, H1N2, H2N2, H3N2, H5N1, H7N2, H7N3, H7N7, H9N2 and H10N7.

In any aspect of the invention, the condition may be caused by a rhinovirus or respiratory syncytial virus (RSV). Further, in any aspect of the invention, the viral mediated exacerbation is rhinovirus or RSV mediated. The rhinovirus or RSV may be any serotype as described herein. Typically, the rhinovirus is a member of the RV-A, RV-B, or RV-C rhinovirus species.

In another aspect of the invention, the condition may be caused viruses of the family/genus influenza, parainfluenza, coronavirus, adenovirus, and metapneumonvirus.

### Treatments, Administration, Dosage and Formulation

The present invention allows identification of individuals who are susceptible to respiratory infections, eg severe lower respiratory tract infections, at an early of life. This provides an opportunity for intervention in the form of pre-emptive therapy.

Exemplary pre-emptive treatments include palivizumab, prednisolone, omalizumab or a polybacterial formulation and any combination thereof.

The term ‘respiratory’ refers to the process by which oxygen is taken into the body and carbon dioxide is discharged, through the bodily system including the nose, throat, larynx, trachea, bronchi and lungs.

As used herein, the upper respiratory tract may include the following regions: nose and nasal passages, paranasal sinuses, the pharynx, and the portion of the larynx above the vocal folds (cords). Typically, the lower respiratory tract includes the following regions: portion of the larynx below the vocal folds, trachea, bronchi and bronchioles. The lungs can be included in the lower respiratory tract and include the respiratory bronchioles, alveolar ducts, alveolar sacs, and alveoli.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. In asthma the FEV_{1.0} (forced expiratory volume in one second) as a percentage of that predicted nomographically based on weight, height and age, may be decreased as may the peak expiratory flow rate in a forced expiration. In COPD the FEV_{1.0} as a ratio of the FVC is typically reduced to less than 0.7. The impact of each of these conditions may also be measured by days of lost work/school, disturbed sleep, requirement for bronchodilator drugs, requirement for glucocorticoids including oral glucocorticoids.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; inhaled corticosteroid (ICS) dose; oral (glucocorticoid) GC usage; need for other medications; need for medical treatment; hospital admission.

The terms “treatment” or “treating” of a subject includes the application or administration of a treatment compound as described herein with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

A positive response to therapy may also be prevention or attenuation of worsening of respiratory symptoms, e.g. asthma symptoms (exacerbation), following a respiratory virus infection. This could be assessed by comparison of the mean change in disease score from baseline to end of study period based on Juniper Asthma Control Questionnaire (ACQ-6), and could also assess lower respiratory symptom score (LRSS symptoms of chest tightness, wheeze, shortness of breath and cough) daily following infection/onset of cold symptoms. Change from baseline lung function (peak expiratory flow PEF) could also be assessed and a positive response to therapy could be a significant attenuation in reduced PEF. For example, a placebo treated group would show a significant reduction in morning PEF of 15% at the peak of exacerbation whilst the treatment group would show a non-significant reduction in PEF less than 15% change from baseline.

The treatments for use according to a method of the present invention is to be administered in an effective amount. The phrase ‘therapeutically effective amount’ or ‘effective amount’ refers to a treatment as described herein that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

The treatments as described herein may be formulated for intranasal administration, including dry powder, sprays, mists, or aerosols. This may be particularly preferred for treatment of a respiratory infection.

Suitable formulations, wherein the carrier is a liquid, for administration, as for example, a nasal spray or as nasal drops, include aqueous or oily solutions of the active ingredient. Alternatively, the treatment may be provided as a dry powder and administered to the upper respiratory tract only as defined herein.

The selection of appropriate carriers depends upon the particular type of administration that is contemplated. For administration via the upper respiratory tract, e.g., the nasal mucosal surfaces, the active compound of the treatments described herein can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of the treatments described herein, various devices are available in the art for the generation of drops, droplets and sprays. For example, a treatment described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

### EXAMPLES

### Example 1—Materials and Methods

### Study Population

Subjects were a subset of 50 individuals from the Childhood Asthma Study, a 10 year prospective birth cohort enrolled prenatally for high risk of asthma development, as described previously (Kusel et al., J Allergy Clin Immunol, 2007, 119:1105-1110; Holt et al., J Allergy Clin Immunol, 2019, 143:1176-1182 e1175; Kusel et al., Pediatr Infect Dis J, 2006, 25:680-686; Kusel et al., Eur Respir J, 2012, 39:876-882; Holt et al., J Allergy Clin Immunol, 2010, 125:653-659; Kusel et al., J Allergy Clin Immunol, 2005, 116:1067-1072). Acute respiratory infections were considered sLRIs if wheeze and/or fever was present in addition to chest rattle. Rattle (chest rattle) was defined as wet noisy breath sounds heard from the child's chest, whereas wheeze was defined wheeze as audible, expiratory, high-pitched whistling sounds. Fever was defined by recording a temperature >38° C. (digital thermometer) on two occasions measured more than 1 hour apart, with 48 hours of the onset of respiratory infection symptoms. Respiratory viral infection histories were determined from detailed assessment and nasopharyngeal aspirates (RT-PCR) collected during home visits within 48 hours of symptom development (Kusel et al., J Allergy Clin Immunol, 2007, 119:1105-1110; Kusel et al., Pediatr Infect Dis J, 2006, 25:680-686). Current wheeze at 5 years (Crwz5) was defined as any wheezy event recorded (parental assessment) in the 12 months before the 5 year follow-up. Asthma at 5 years was defined as having doctor diagnosis of asthma ever, a prescription to asthma medication, and current wheeze at 5 years. A non-asthmatic determination at 5 years had none of these criteria. Umbilical cord blood was collected at birth and peripheral blood was collected at 0.5, 1, 2, 3, 4, 5, and 10 years (as close to the birth date as possible).

### Immunophenotyping

Cryopreserved CBMCs were thawed and washed with RPMI 1640 (Gibco) containing 10% non-heat-inactivated FBS (Serana Australia). 10 μl of cell mixture was stained with trypan blue and counted with a haematocytometer. Approximately 1×10⁶ cells were aliquoted for immunophenotyping, and 0.25×10⁶ cells were aliquoted for unstained controls, for each sample. Cells were pelleted by centrifugation at 1500 rpm (˜500 g) for 5 minutes at 4° C., and excess media was removed by vacuum aspiration. Each sample was incubated with 50 μl of a master mix of monoclonal antibodies (CD19-FITC RRID: AB_395812, CD3-AF700 RRID: AB_396952, CD4-V500 RRID: AB_1937323, CD14-APC-Cy7 RRID: AB_1645464, HLA-DR-PerCP-Cy5.5 cat #347364, CD25-BV421 RRID: AB_11154578, CD127-BV605 RRID: AB_2738138, CD123-CF594 RRID: AB_11153664, CD11c-PE-Cy7 RRID: AB_10611859 [BD Bioscience] and FcεRIα-APC RRID: AB_10671394 [eBioscience]) in cold FACS Buffer (PBS+1% BSA) for 30 minutes at 4° C. in the dark. Cells were washed, fixed and permeabilized with (Cytofix/Cytoperm buffer (BD Biosciences)) for 1 hour, and incubated for 30 minutes with FoxP3-PE (intra-cellular, BD Biosciences). The same antibody batches were used for all samples at the manufacturers recommended dilution. Individual cells were acquired using the LSR-Fortessa platform with FACSDiva software (BD Biosciences) following quality control assessment (Rainbow calibration and CS&T beads (BD Biosciences) prior to each cytometry run and unstained control were included for each sample. Initially, samples were compensated and gated with FlowJo 10.3 software. Compensated FCS files were imported into the R (3.6.2) statistical environment and pre-processed with the flowWorkspace and flowCore packages. Logicle transformation (flowCore) and batch correction (sva) was applied to all samples. Nonparametric paired (Wilcoxon signed rank test) or unpaired (Mann-Whitney U test) tests were used to determine between group differences.

### In Vitro Cell Culture

Samples were assigned randomized blocks and cultured sequentially by the same personnel using consistent reagent/stimuli stocks. Cord blood erythrocytes were immunomagnetically depleted (EasySep kit, StemCell) and each sample was cultured in RPMI+5% AB serum (non-heat inactivated, Sigma-Aldrich) for 18 hours (37° C., 5% CO₂) with LPS (Enzo Biochem, 1 ng/ml), Imiquimod (Invivogen, 5 μl/ml) and Poly(I:C) (Invivogen, (50 μl/ml), alongside matched unstimulated controls. Aliquots of culture supernatant were stored at −20° C. for cytokine quantification. Cell pellets were stored in Trizol (Invitrogen) at −20° C. for RNA extraction.

### Data Generation

RNA-Seq: RNA was extracted with RNeasy MinElute Kits (Qiagen) in batches and the extraction batch information was recorded. RNA concentration was measured (Bioanalyzer; Agilent, Santa Clara, USA) and found to be good quality (RIN score; mean=8.514, 95% CI=8.46-8.567). A low yield protocol was employed with sequencing libraries prepared with NEBNext Ultra II Kits (New England BioLabs, Massachusetts, USA) and sequenced with the NovaSeq 6000 (Illumina, San Diego, USA) platform at the Australian Genome Research Facility (AGRF, Melbourne, Australia) for sequencing (100 bp paired-end).

Cytokines: The concentrations of 48 cytokines (Bio-plex Pro, BioRad) were simultaneously quantified with the Luminex 200 system (Luminex). Analyte quantification (pg/ml) was determined by alignment to a standard curve. The cytokine panel included CTACK, FGF basic, Eotaxin, G-CSF, GM-CSF, GRO-α, HGF, IFN-α2, IFN-γ, IL-1β, IL-1ra, IL-1α, IL-2, IL-2Rα, IL-3, IL-4, IL-5, IL-6, IL-7, IL-8, IL-9, IL-10, IL-12(p40), IL-12(p70), IL-13, IL-15, IL-16, IL-17A, IL-18, IP-10, LIF, MCP-3, MCP-1, M-CSF, MIF, MIG, MIP-1α, MIP-1β, β-NGF, PDGF-BB, SCF, SCGF-β, SDF-1α, RANTES, TNF, TRAIL, and VEGF. Nine cytokines were outside the limit of detection in >20% of samples and were removed.

### Data Pre-Processing

RNAseq: The binary base call (BCL) sequence files were converted to fastq files with the bcl2fastq pipeline (Illumina). Sequence data were processed with MEdical Sequence Analysis Pipeline (MESAP) and aligned to the hg38 genome with HISAT2 (Pertea et al., Nat Protoc, 2016, 11:1650-1667) and counts were quantified with summariseOverlaps function from the GenomicAlignments R package. Pre- and post-alignment QC was assessed with FastQC and SAMStat, respectively.

Cytokines: Cytokines were excluded if more than 30% of sample (excluding unstimulated samples) recorded out of range (OOR) values. This resulted in the removal of CTACK, IL-3, IL-7, IL-8, IL-13, IL-18, PDGF-BB, SCGFβ, SDF-1α from further analysis. Remaining OOR values were imputed below/above the minimum/maximum based on truncated normal distribution with the rtruncnorm function from the truncnorm R package, so that partial information is used to define values below/above the limit of detection for imputation. The most appropriate method of transformation and normalisation was tested (data not shown), and ArcSinh transformation and Loess normalisation was applied. Batch effects were removed with linear modelling (removeBatchEffect function from the limma R package).

Dimensionality reduction: The experimental design ensured that matched data was generated from 4 conditions (Unstimulated, LPS-, Imiquimod-, and Poly(I:C)-stimulated) for each individual within the same batch. This allows for a multi-level design for dimensionality reduction analysis (Principal Component Analysis), whereby the within subject variance is decomposed from the between subject variance, which considerably improves the power and interpretability of subsequent multivariate analysis. For this purpose, the withinVariation function was adapted from the mixOmics package in R (Rohart et al., PLoS Comput Biol, 2017, 13: e1005752).

Transcripts: Following pre-processing, CBMC gene expression data for matched Unstimulated, LPS-, Imiquimod-, and Poly(I:C)-stimulated samples for the 50 individuals was filtered to reduce noise, to only significantly variable genes, with the varianceBasedfilter function in R. For this analysis, genes were considered significant with a p value lower than a strict threshold of 2.88×10⁻⁶, determined by 0.05/number of genes (n=17356). This resulted in 5,885 genes for dimensionality reduction. Within subject variation was calculated with the withinVariation function, genes were scaled to unit variance and the PCA function from the FactoMineR package was used for principal component analysis (Lê et al., J of Statistical Software, 2008, 25: 1-18) and the principle component scores and variable contributions were used for plots.

Cytokines: Following pre-processing, within subject variation was calculated (as above) from the CBMC cytokine concentration data (n=39) for matched Unstimulated, LPS-, Imiquimod-, and Poly(I:C)-stimulated samples for the 50 individuals. Principal component analysis was applied as described above.

### Transcriptomic Analysis

EdgeR: A total of 50,019 raw transcripts were available for analysis following pre-processing. Raw transcripts were removed if they had no counts in any sample, lacked annotation, or had <0.5 counts per million in s 25 samples. This strategy produced 17,363 transcripts for analysis. Data was normalised with the trimmed mean of M-values (TMM normalisation (Robinson et al., Genome Biol, 2010, 11: R25)). As the experimental design involved block randomisation (with respect to age and stimuli) into batches, there was no batch effect related to cell culture batch number for these paired comparisons. There was no discernible batch effect observed for unpaired comparisons, however culture batch was included as covariate for unpaired analysis (i.e. sLRI susceptibility in infancy) regardless. Unwanted variation was identified and removed with the RUVg function from the RUVSeq R package (Risso et al., Nat Biotechnol, 2014, 32: 896-902), which models a set of empirical control genes (not significantly different between any comparison of interest) to determine putative technical effects (lane, sequencing, etc). A paired designed was employed for analysis between matched stimulated and unstimulated samples, which included trends identified by RUVg as covariates. The EdgeR (Robinson et al., Bioinformatics, 2010, 26: 139-140) pipleline was run with default parameters, which includes the estimateDisp, glmQLFit, glmLRT, and topTags, which fits a negative binomial generalized log-linear model to the counts for each gene and conducts genewise likelihood ratio tests. For analysis between CBMC and matched samples at age 5 years (n=27), a paired design was employed which modelled differences between matched unstimulated and the corresponding simulated samples, as well as RUVg trends. An unpaired design which modelled unstimulated/stimuli and RUVg trends was used to determine differences between the primary outcome. Genes were considered significantly different if it recorded an FDR-adjusted Benjamini-Hochberg p value <0.01 and a Log 2 fold change above 1 (upregulated) or below −1 (downregulated). A matrix of corrected gene counts was generated for downstream network analysis. Size factors were estimated using the median ratio method with the estimateSizeFactors function and variance stabilizing transformation (VST) was applied to the count data with the varianceStabilizingTransformation function, from the DESeq2 package (Love et al., Genome Biol, 2014, 15: 550). RUVg trends which related to technical variation and culture batch were removed as covariates with a linear model with the removeBatchEffect function from the limma package.

Limma-voom: Transcript filter, normalisation and model design was performed the same as described for EdgeR analysis. The data was transformed to log 2-counts per million and the mean-variance relationship was estimated to produce weights using the voom function. The limma (Ritchie et al., Nucleic Acids Res, 2015, 43: e47) pipeline was run with default parameters, which includes the ImFit, contrasts.fit, eBayes, and topTable functions. The same criteria as the EdgeR analysis was applied to determine gene significance. From this analysis, the moderated t-statistic calculated for each gene was plotted by module for each network, as a way of displaying which modules are differentially regulated between simulated and matched unstimulated samples. The moderated t-statistic is the ratio of the M-value (log 2-fold change) to its standard error, which has been “moderated” (empirical Bayes) across all genes. Applying the module eigengene instead of the moderated t-statistic yielded the same overall result with respect to module up-/down-regulation (data not shown). Modules with medians above a moderated t-statistic of 2 are considered significantly upregulated and those below −2 are considered significantly downregulated.

Weighted Gene Co-expression Network Analysis: Corrected count data (described above) were used as input which included 17,363 genes for analysis. For this analysis, three perturbation networks were created, each of which included unstimulated samples and the corresponding stimulated samples (i.e. the LPS, Imiquimod, and Poly(I:C) networks) (WGCNA (Zhang et al., Stat Appl Genet Mol Biol, 2005, 4: Article 17; Langfelder et al., BMC Bioinformatics, 2008, 9: 559)). The varianceBasedfilter function was used to filter significantly variable genes (p value <0.01) for each condition, and union genes between the unstimulated and respective stimulated samples. This strategy resulted in 6561, 6757, and 6764 genes available for the LPS, Imiquimod, and Poly(I:C) networks, respectively. Soft powers were calculated with the pickSoftThreshold function with the networkType parameter set to “signed”. This resulted in soft powers of 7, 8, and 7 for the LPS, Imiquimod, and Poly(I:C) networks, respectively. Adjacency and topological overlap matrices (TOM) were created with the adjacency and TOMsimilarity functions, respectively, with “signed” network specified. The TOM dissimilarity matrices were calculated by one minus the TOM similarity matrix (1-TOM). Modules were identified by hierarchical clustering with the hclust function (method=“average”) and pruned with the cutreeDynamic function (method=“hybrid”, deepSplit=2, minClusterSize=50). TOM plots were created with the TOMplot function. Module eigengenes were calculated with the moduleEigengenes function. Modules were merged if they were similar, determined by correlation of their eigengenes, hierarchical clustering, and dendrogram cut at 0.1 with the mergeCloseModules function. Network statistics and within module connectivity was calculated with the intramodularConnectivity function. Modules were annotated with a consensus approach by assessing significantly enriched pathways from: Gene Ontology term enrichment (GOenrichmentAnalysis), ReactomePA (Yu et al., Mol Biosyst, 2016, 12: 477-479) and clusterProfiler (Yu et al., Omics, 2012, 16:284-287) R packages, InnateDB (Breuer et al., Nucleic Acids Res, 2013, 41: D1228-1233), and identification of top module genes (Log₂-FC/gene connectivity). Module preservation between networks was calculated with the modulePreservation function, with 200 permutations, networkType set to “signed”, and the “gold” (random) module size set to the average module size for each comparison. The ranked expression was calculated as the (rank) average expression of each genes across all samples, and ranked connectivity was calculated with the (rank) softConnectivity function, with type set to “signed” and power set to the corresponding network's soft power. Soft connectivity is defined as the sum of the adjacency (co-expression measure) of each gene in the network to all other genes. Connectivity density were determined with the density function and the Sheather-Jones smoothing bandwidth method was used. Connectivity densities were assessed for normal distribution with a Lilliefors test of normality (lillie.test function). A Spearman's correlation matrix was also calculated for each module to separately assess intramodule connectivity, defined as the sum of the correlation value of each gene to all other genes. Network wiring diagrams of the top 20 most connected genes were constructed with the graph_from_adjacency_matrix function from the igraph R package. Node size represents number of connections (degree) among the total network and edge with indicates strength of connection (red edges denote a correlation >0.8).

### Master Regulator Analysis

A gene regulator network was reverse engineered with ARACNe (Margolin et al., BMC Bioinformatics, 2006, 7 Suppl 1, S7) and transcription factor activity was inferred with VIPER (Alvarez et al., Nat Genet, 2016, 48:838-847). Significant (p<0.05) TFs were considered drivers of the responses if they had known binding motifs in the region of regulon target genes determined by RcisTarget (Aibar et al., Nat Methods, 2017, 14:1083-1086). Normalised expression scores (NES) outputted from VIPER were retained for downstream analysis.

### Machine Learning

Gene expression data was randomly assign into training (50%) and validation (50%) sets and filtered to only the respective module genes for each analysis. The same random assignment was applied for all models. For validation models, CAS cohort data was filtered to respective module genes and used as the training set, and the external gene expression data was used for validation (filtered to identical input genes).

The RandomForest package was used in R for model construction, the number of decision trees (ntree) and candidate variables (mtry) were optimized according to the out-of-bag error rate. Random Forest (RF) analysis was performed using the modules defined by WGCNA, which by design clusters genes according to co-expression, so that module member genes exhibit high multicollinearity, which is a recognised influence on RF interpretation (Strobl et al., BMC Bioinformatics, 2008, 9: 307; Tolosi et al., Bioinformatics, 2011, 27: 1986-1994). However, collinearity primarily affects the interpretation of variable importance, and not the overall model prediction accuracy. For this reason, the RF classifiers used in the present study were employed principally to test the utility of IFN module genes to predict outcomes, and the variable importance measures (whilst reported in the figures), should be considered an underestimation of their true value.

CAS cohort: To account for potential differences in baseline/unstimulated CBMC gene expression between individuals, A values were taken from the matched stimuli gene expression profiles (e.g. LPS-stimulated gene expression—matched unstimulated gene expression=adjusted LPS-stimulated gene expression matrix), and these were used as input. Genes were filtered to only those present in the IFN modules of the corresponding response. Subjects (n=50) were randomly assigned to either a test or validation set (50/50 split), and the same random assignment was applied to the LPS, Imiquimod and Poly(I:C) datasets. The randomForest function (randomForest R package) was to optimise each RF model, with respect to the number of variables randomly sampled as candidates at each split (“mtry”) and the number of decision trees to grow (“ntree”), to classify individuals who did and did not experience an sLRI in infancy. For the mtry parameter, a sequence from the lower of 10 or the square root of the number of input genes up to five times the square root of the number of input genes, by an increase of one, was defined. RF classifiers were built for all numbers in the sequence, with ntree set to 1000, and the mtry value which recorded the lowest out-of-bag error rate (OOBer) was selected as optimal. The lowest number was selected in the case of a tie in OOBer. For the ntree parameter, a sequence from 500 to 10,000 increasing in increments of 100 was defined, and RF classifiers were built for all numbers in the sequence, with mtry set to the optimal as defined above. The optimal ntree was selected that which produced the smallest OOBer (lowest number if a tie). This approach resulted in a mtry of 11, 23, and 121 and a ntree value of 5400, 1000, and 1000 for the LPS, Imiquimod, and Poly(I:C) RF models, respectively. The trained models, which internally bootstrap the training set (70/30 split) reported OOBer of 36%, 72%, and 52% for the LPS, Imiquimod, and Poly(I:C) RF models, respectively. Following optimisation, final RF classifiers were used to predict the primary outcome status in the corresponding validation set (without class labels) with the predict function (stats R package). The prediction and performance functions from the ROCR R package were used to compare the predictions to true values and to determine true and false positive rates to calculate and the area under the Receiver Operating Characteristic (ROC) curve.

Training/validation set re-sampling: To test the reproducibility of the RF classifiers, the adjusted LPS, Imiquimod, and Poly(I:C) datasets were each randomly re-sampled 2000 times, with respect to their training/validation set assignment (50/50 split). The same 2000 random assignments were used for the LPS, Imiquimod, and Poly(I:C) classifiers. RF classifiers were built on the training set and tested on the validation set (as above) for each re-sample (using the previously determined optimal parameters, above), and the area under the ROC curve was recorded each time to determine the prediction accuracy. To assess whether different proportional assignment of training and validation set produced similar results, RF classifiers of the adjusted LPS, Imiquimod, and Poly(I:C) datasets were each randomly re-sampled 1000 times (with respect to their training/validation set assignment) at training/validation assignments of 60%/40% and 70%/30%. The same 1000 random assignments were used for the LPS, Imiquimod, and Poly(I:C) classifiers, and they were built with the above (optomised) parameters. The area under the ROC curve was recorded for each re-sample to determine the prediction accuracy.

Validation in external cohorts: We trained RF classifiers on our CBMC data and used them to classify samples derived from a series of publicly available data sets from the Gene Expression Omnibus. In general, we used unstimulated samples from the CAS cohort to represent “healthy” individuals (absence of infection), and stimulated samples to represent anti-bacterial (LPS) and anti-viral (Imiquimod/Poly(I:C)) innate immune responses of infants/children with confirmed infection. GSE72809: RF classifiers were trained on the adjusted LPS- and Imiquimod-/Poly(I:C)-stimulated CBMC gene expression datasets (n=50 each) and used to predict children hospitalised with bacterial (n=52) and viral (n=92) infections, respectively, from healthy controls (n=52), from blood-derived gene expression profiles. Model optimization was implemented and the model prediction accuracy was tested on the external gene expression profiles as previously described (above). GSE113211: RF classifiers were trained on the adjusted Imiquimod- and Poly(I:C)-stimulated CBMC gene expression datasets (n=50 each) and used to predict infants (s 18 months, n=15) and young children (18 mo-5 yrs, n=16) hospitalised with acute viral bronchiolitis from matched samples collected post-convalescence (symptom-free, 8.8±2.5 weeks post-infection) from PBMC samples. Model optimization was applied and the model prediction accuracy was tested on the external gene expression profiles, as previously described (above), for all subjects together as well as infants and children separately. GSE115770: RF classifiers were trained on the adjusted Imiquimod- and Poly(I:C)-stimulated CBMC gene expression datasets (n=50 each) and used to predict study visits asthmatic children (6-17 yrs) with viral-associated (n=193) “cold”-like illness from those with non-viral “cold”-like illness (n=105) (samples taken 1-6 days post-onset), some of which later experienced exacerbations (58 did and 25 did not). Model optimization was implemented and the model prediction accuracy was test as previously described (above), for viral infection given cold symptoms, and viral infection given exacerbation, for nasal- and blood-derived gene expression profiles separately.

### Multi-Omic Data Integration

A DIABLO (Singh et al., Bioinformatics, 2019, 35:3055-3062) model constructed for supervised multi-omic data integration, which generalizes Partial Least Squares analysis to maximize co-expression between matched datasets. All datasets (except immunophenotyping) were baseline adjusted prior to analysis, and gene expression data was filtered to significantly variable genes (n=6353) to reduce noise. Number of components and feature selection parameters were tuned with 5× cross validation.

### Statistical Analysis

All statistical analysis was computed in the R environment (version 3.6.2) and graphs were produced from R or Prism software (version 8, GraphPad Software, La Jolla California USA). Non-parametric statistical methods were applied to test group differences (Mann-Whitney U test (unpaired analysis) and Wilcoxon signed Rank Test (paired analysis); wilcox.test function [stats R package]) and correlations (Spearman's rank correlation coefficient; cor.test function [stats R package]). For comparisons of study population characteristics, Fisher's Exact test (fisher.test function [stats R package]) was used to calculate odds ratios, 95% Confidence Intervals, and accompanying P values for categorical variables, and Mann-Whitney U test was used to determine p values for continuous variables.

### Example 2—Study Population Clinical Characteristics

The study population consisted of a subset of 50 children within the Childhood Asthma Study (CAS) cohort. 23 subjects (46%) experienced at least one wheezy and/or febrile sLRI in their first year (infancy) and this was the primary outcome of interest (Table 1). Current wheeze (OR=2.48) and asthma (OR=2.86) prevalence at 5 years of age were higher for susceptible individuals but this was not statistically significant. No difference was observed with respect to sex, gestational weeks, birth weight, skin prick test positivity, and URIs in infancy for the primary outcome. Overall, this subset was found to be representative of the CAS cohort (n=263) with respect to key clinical characteristics (Table 1). Rhinovirus was the most frequent viral agent identified from the first year of life in this subset (present in 56.9% of infectious nasopharyngeal samples) followed by RSV (13.125%) (data not shown).

TABLE 1
Characteristics and representation of the study population

| | CAS subset | | sLRly1 negative | | sLRly1 positive | | OR | |
|---|---|---|---|---|---|---|---|---|
| | (n = 50) | | (n = 27) | | (n = 23) | | (95% CI) | |
| Sex (female) | 24/50 | (48%) | 14/27 | (51.85%) | 10/23 | (43.48%) | 0.72 | (0.2-2.51) |
| Gestation (weeks; mean [range]) | 39.14 | [36-41] | 39.11 | [36-41] | 39.17 | [37-41] | NA | |
| Birth weight (grams; mean | 3496.52 | [2755-4415] | 3468.56 | [2755-4270] | 3529.35 | [2920-4415] | NA | |
| [range]) | | | | | | | | |
| SPT+ at 0.5, 2, or 5 years | 24/50 | (48%) | 16/27 | (59.26%) | 14/23 | (60.87%) | 1.07 | (0.3-3.88) |
| URI in first year | 47/50 | (94%) | 26/27 | (96.3%) | 21/23 | (86.96%) | 0.41 | (0.01-8.41) |
| LRI in first year | 39/50 | (78%) | 16/27 | (59.26%) | 23/23 | (100%) | Inf | (2.97-inf) |
| sLRly1 in first year | 23/50 | (46%) | 0/27 | (0%) | 23/23 | (100%} | Inf | (77.85-Inf) |
| Current wheeze at 5 years | 14/43 | (32.56%) | 6/25 | (24%) | 8/18 | (44.44%) | 2.48 | (0.57-11.49) |
| Asthma at 5 years | 9/43 | (20.93%) | 3/19 | (15.79%) | 5/14 | (35.71%) | 2.86 | (0.44-22.97) |
| Never wheeze (years 3-5) | 15/44 | (34.09%) | 17/24 | (70.83) | 12/18 | (66.67%) | 0.83 | (0.18-3.82) |

| | P | CAS cohort | | OR | | P |
|---|---|---|---|---|---|---|
| | value | (n = 263) | | (95% CI) | | value |
| Sex (female) | 0.58 | 115/251 | (45.82%) | 0.92 | (0.48-1.77) | 0.88 |
| Gestation (weeks; mean [range]) | 0.96 | 39.03 | [34-41] | NA | | 0.89 |
| Birth weight (grams; mean | 0.65 | 3406.17 | [2085-5110] | NA | | 0.27 |
| [range]) | | | | | | |
| SPT+ at 0.5, 2, or 5 years | 1 | 118/198 | (59.6%) | 1.59 | (0.82-3.13) | 0.15 |
| URI in first year | 0.59 | 215/235 | (91.49%) | 0.69 | (0.13-2.46) | 0.78 |
| LRI in first year | 0.0004 | 160/235 | (68.08%) | 0.6 | (0.26-1.28) | 0.18 |
| sLRly1 in first year | >0.0001 | 101/235 | (42.98%) | 0.88 | (0.46-1.72) | 0.75 |
| Current wheeze at 5 years | 0.2 | 56/198 | (28.28%) | 0.82 | (0.38-1.8) | 0.58 |
| Asthma at 5 years | 0.24 | 37/198 | (18.69%) | 0.87 | (0.37-2.24) | 0.83 |
| Never wheeze (years 3-5) | 1 | 70/198 | (35.35%) | 1.06 | (0.51-2.72) | 1 |

Abbreviations: CAS-Childhood Asthma Study, OR = Odds Ratio, CI = Confidence Interval, URI = Upper respiratory Infection (viral), LRI = Lower respiratory Infection (viral).
sLRly1 represents the primary outcome (sLRI incidence in the first year of life).
For categorical variables, odds ratios, 95% CIs and accompanying P values were determined by Fishers Exact test.
For continuous variables, P values were determined by Mann-Whitney U test.
P values represented in bold are consider statistically significant.

### Example 3—Baseline Flow Cytometry

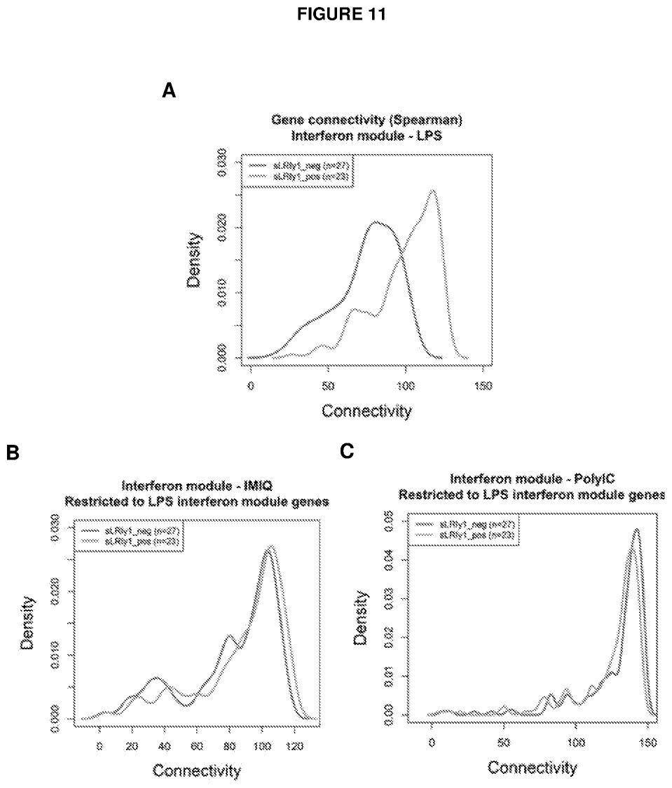

An 11-colour flow cytometry panel was applied to baseline cord blood mononuclear cell (CBMC) samples to assess cellular composition. Lymphocytes (T and B cells) composed the majority of cell types identified among CBMC (FIG. 11B). CD14+ monocytes and conventional dendritic cells (cDCs) were identified among the myeloid compartment, and smaller proportions of plasmacytoid DCs (pDC) and basophils were also identified (FIG. 11B). There was no difference in baseline cellular composition with respect to sLRI in the first year of life (data not shown).

### Example 4—Multi-Omic Profiling of Innate Immune Responses in CBMC

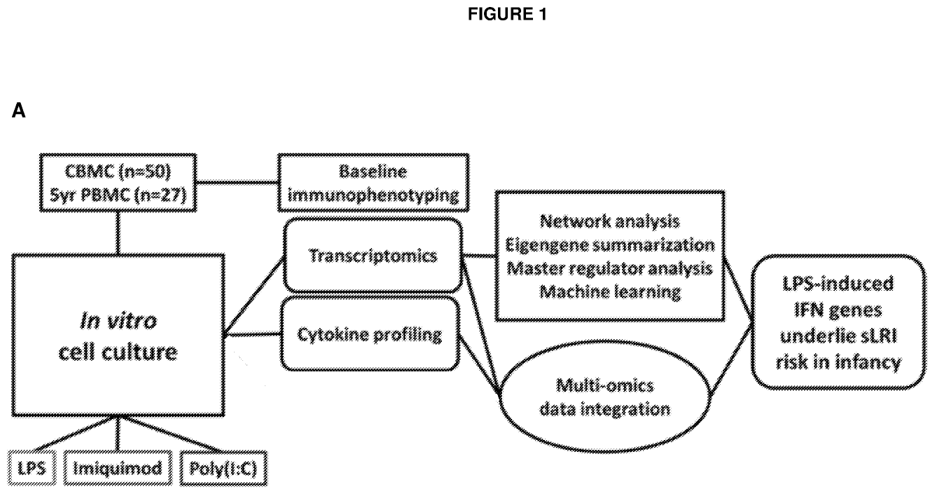

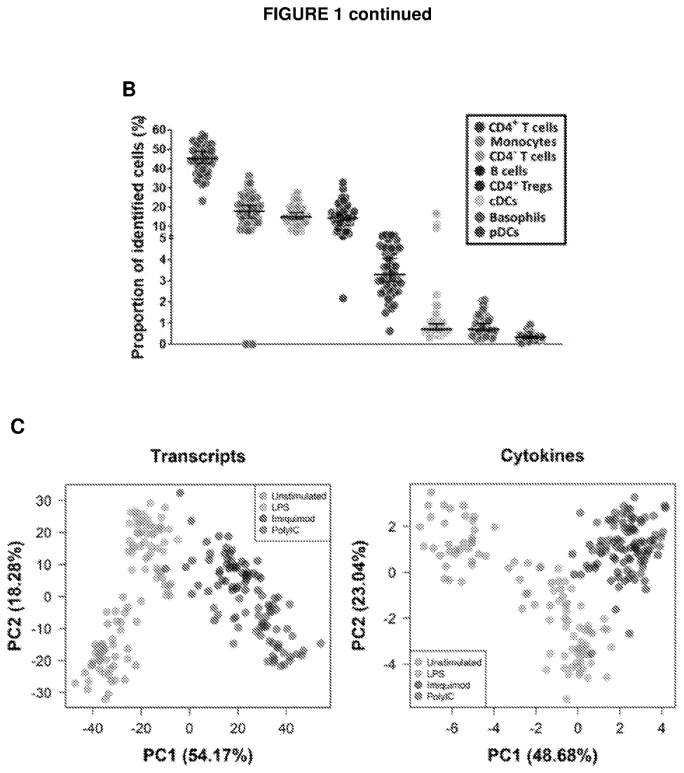

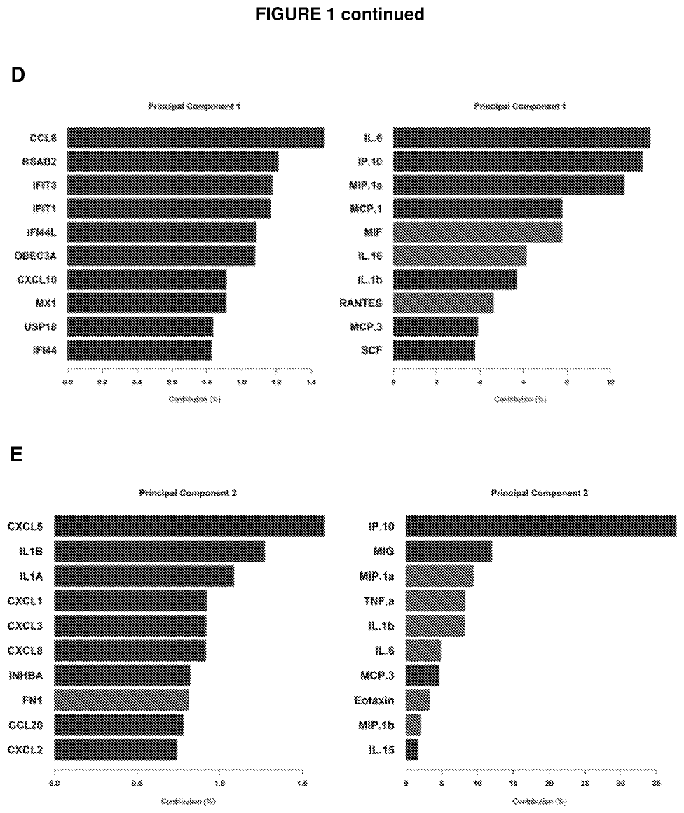

CBMC from all 50 subjects were cultured for 18 hours with LPS (TLR4), or Imiquimod (TLR7), or Poly(I:C) (TLR3) to trigger innate immune responses, along with unstimulated controls (FIG. 1A). This timepoint was selected to capture signalling cascades downstream of the immediate and secondary response programs (Shalek et al., Nature, 2014, 510:363-369; Jovanovic et al., Science, 2015, 347:1259038; Lawlor et al., Front Immunol, 2021, 12:636720). Gene expression was profiled from cell pellets (RNAseq) and supernatants were used to profile cytokines (Multiplex assay). Matching PBMC samples collected at age 5 were available for a subset of the subjects (n=27), and these were cultured in parallel under the same conditions. Following data pre-processing and filtering, 17,363 transcripts and 39 cytokines were available for analysis (see Example 1 for methods). An unsupervised Principal Component Analysis (PCA) dimension reduction was applied for exploratory data analysis. The samples from each omic layer clustered by stimuli as expected (FIG. 1C). For transcripts and cytokines, the first two principal components captured interferon (IFN) and proinflammatory features (e.g. CXCL10/IP-10, IL-1β, IL-6) (FIG. 1D).

### Example 5—IFN and Proinflammatory Gene Expression Programs are Upregulated in CBMC Responses

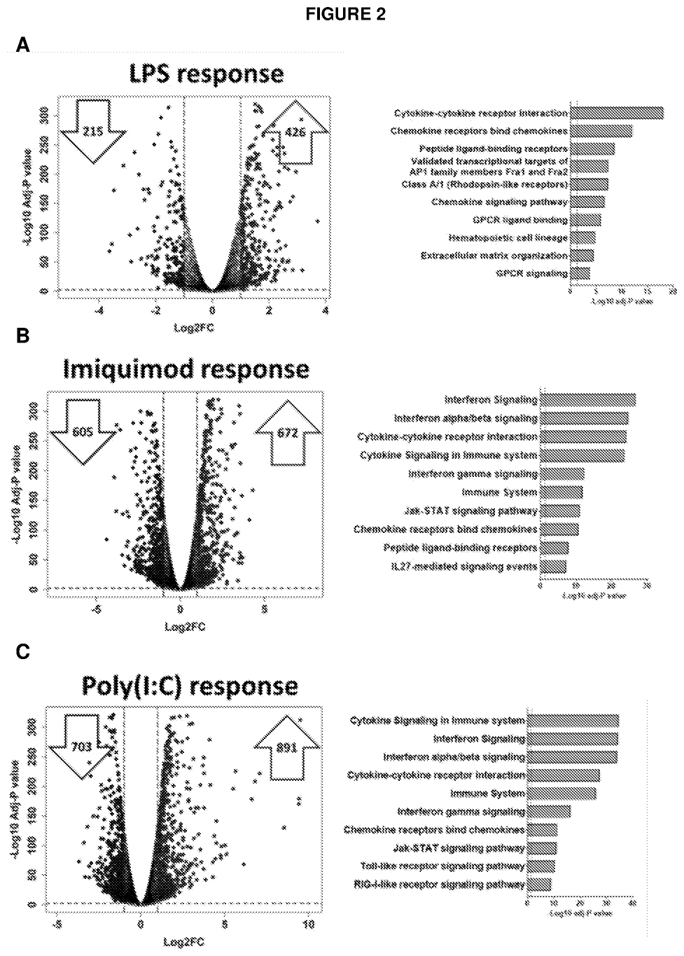

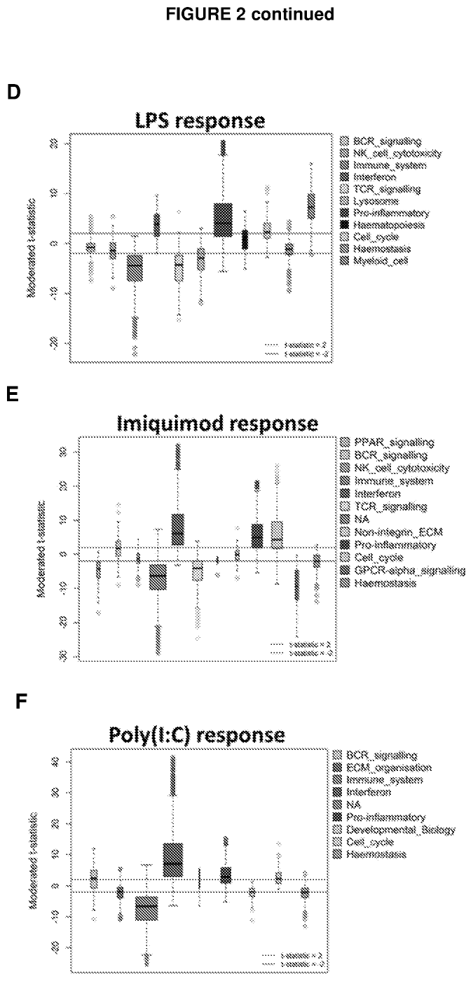

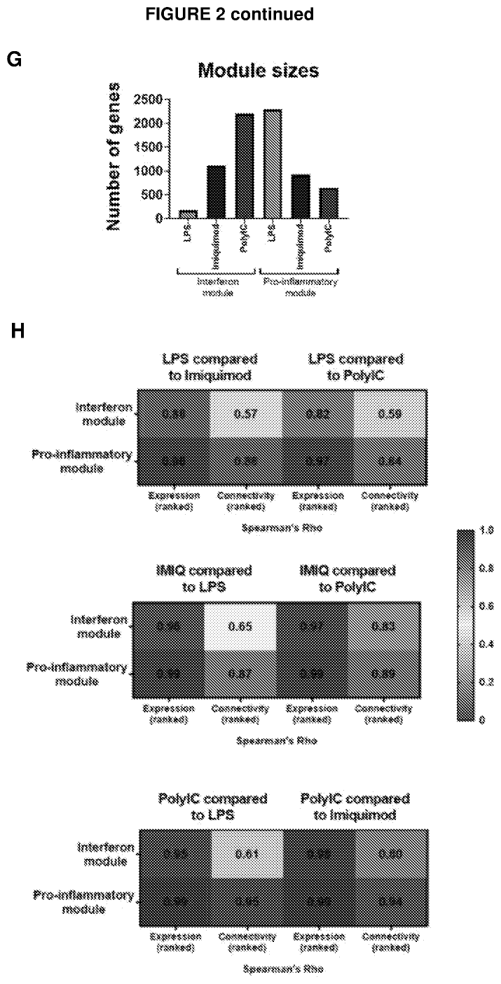

The first analyses were focused on the transcriptomics data as these data provide genome-wide coverage. Employing differential expression analysis, 641 differently expressed genes (DEGs) were identified for the cord blood LPS response (Log 2-fold change >1, adjusted-P value <0.01), and greater than 1000 DEGs for the imiquimod and Poly(I:C) responses (FIG. 2A-C; left panel). Pathways analysis (InnateDB; Breuer et al., Nucleic Acids Res, 2013, 41:D1228-1233) identified an enrichment of cytokine and chemokine signalling pathways from upregulated genes in all responses, and IFN signalling pathways were prominent for imiquimod and Poly(I:C) CBMC responses (FIG. 2A-C; right panel). Notably, the viral stimuli triggered a common set of 429 upregulated genes and this constituted a core antiviral response shared between TLR3 and TLR7 activation (data not shown). In addition, 462 and 243 genes were identified that were specifically upregulated in response to Poly(I:C) or Imiquimod respectively, demonstrating unique signalling pathways downstream of TLR3 or TLR7 activation (data not shown). Next CIBERSORTx was employed to estimate the post-culture cellular composition from the RNA-Seq data (Newman et al., Nat Biotechnol, 2019, 37:773-782). Prominent cell types included monocytes, B cells, and CD4+ T cells (data not shown). The erythrocyte proportion was negligible as a result of immunomagnetic depletion (see Example 1 for methods). Cell composition changes were identified between stimuli and age, but not sequence order, sex, or sLRI encounter in infancy. Further investigations for variations in innate immune gene expression was also conducted in the matching samples collected at birth versus age 5 (n=27 per age/stimuli) (data not shown). Interestingly, the LPS response at 5 years was characterised by upregulation of IFN-related genes, including IRF1, STAT1, and IFIT1-3, compared to birth (Table 2). In contrast, differential expression of IFN-related genes was not observed between birth and age 5 following imiquimod or Poly(I:C) stimulation (data not shown). Finally, no genes were significantly different between individuals resistant and susceptible to sLRIs in infancy for any condition from this analysis (data not shown), suggesting that sLRI risk is not conferred by individual gene expression magnitude alone.

TABLE 2
Differential expression of IFN-related genes at birth and
5 years following LPS stimulation.

| Symbol | logFC | FDR |
|---|---|---|
| IFNG | −1.841 | 3.45E−28 |
| IFIT3 | −1.779 | 3.29E−166 |
| TLR3 | −1.644 | 1.93E−16 |
| IFIT1 | −1.566 | 1.78E−75 |
| OASL | −1.482 | 7.886−58 |
| IFIT2 | −1.232 | 4.03E−82 |
| MX1 | −1.097 | 3.56E−107 |
| OAS1 | −1.097 | 8.108−79 |
| STAT1 | −1.045 | 1.48E−89 |
| IRF1 | −1.044 | 3.05E−97 |

### Example 6—Identification of Co-Expression Networks Underlying Innate Immune Function

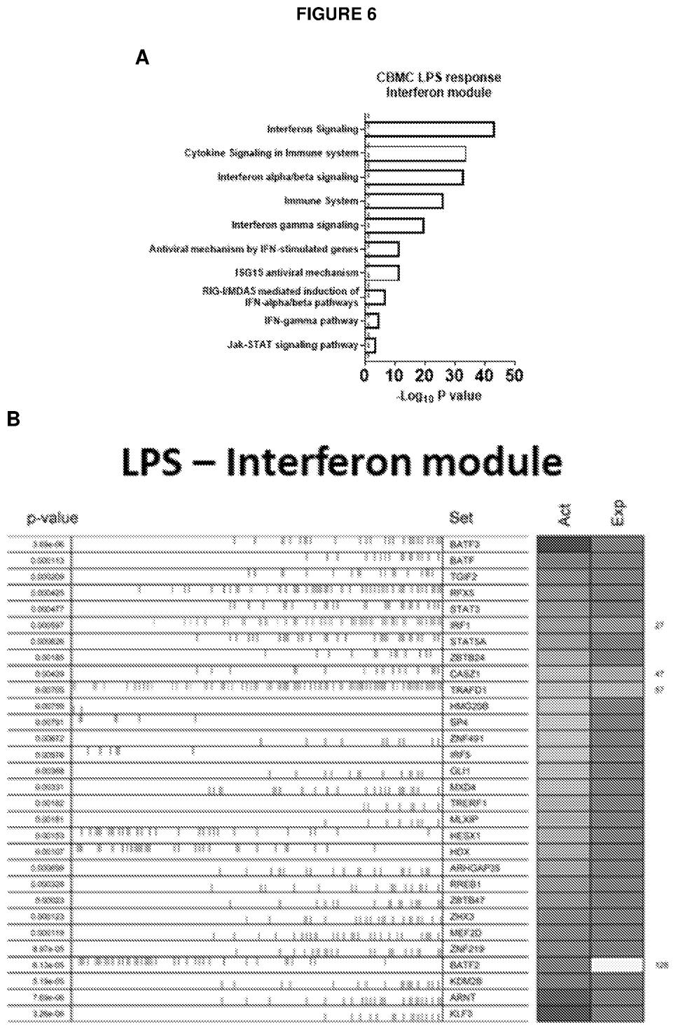

Genes do not function in isolation, they work together in networks. A weighted gene co-expression network analysis (WGCNA) was employed to elucidate the global connectivity structure and functional organisation of gene expression. This analysis identified 11, 11, and 8 co-expression modules for the LPS, Imiquimod and Poly(I:C) responses, respectively (FIG. 2D-F). All responses exhibited upregulation of IFN and proinflammatory modules, and as the integral components of the cord blood innate responses had already been identified, they were therefore carried forward for downstream analysis (FIG. 2D-F, FIG. 6A). The LPS response had the smallest IFN module (180 genes; Table 3) compared to Imiquimod (1114 genes) and Poly(I:C) (2201 genes) and the inverse was true of the proinflammatory modules (LPS, 2297 genes; Imiquimod, 924 genes; Poly(I:C), 646 genes) (FIG. 2G). Notably, there was substantial overlap between IFN and proinflammatory module genes of different stimuli, particularly between the Poly(I:C) IFN and LPS proinflammatory modules (n=385 genes) (data not shown).

TABLE 3
LPS-induced interferon module genes. Gene symbol and ENSEMBL accession
numbers are provided.

| | | | | | |
|---|---|---|---|---|---|
| IDO1 | SOCS1 | GBP4 | CD80 | GBP1 | CXCL11 |
| ENSG00000131203 | ENSG00000185338 | ENSG00000162654 | ENSG00000121594 | ENSG00000117228 | ENSG00000169248 |
| CXCL9 | MT2A | TCAF2 | STAT1 | RTP4 | GBP5 |
| ENSG00000138755 | ENSG00000125148 | ENSG00000170379 | ENSG00000115415 | ENSG00000136514 | ENSG00000154451 |
| FBXO6 | HAPLN3 | CCL19 | APOL1 | PMAIP1 | USP30-AS1 |
| ENSG00000116663 | ENSG00000140511 | ENSG00000172724 | ENSG00000100342 | ENSG00000141682 | ENSG00000256262 |
| IL15RA | GBP2 | PARP9 | IRF1 | TCAF2C | TNFSF10 |
| ENSG00000134470 | ENSG00000162645 | ENSG00000138496 | ENSG00000125347 | ENSG00000283528 | ENSG00000121858 |
| APOL3 | TCAF2P1 | EPSTI1 | UBE2L6 | ETV7 | TTC39A |
| ENSG00000128284 | ENSG00000159860 | ENSG00000133106 | ENSG00000156587 | ENSG00000010030 | ENSG00000085831 |
| CETP | XAF1 | CMPK2 | GBP1P1 | HAS2 | APOL2 |
| ENSG00000087237 | ENSG00000132530 | ENSG00000134326 | ENSG00000225492 | ENSG00000170961 | ENSG00000128335 |
| IFI44 | WARS1 | RSAD2 | OR211P | LGALS3BP | IFNG |
| ENSG00000137965 | ENSG00000140105 | ENSG00000134321 | ENSG00000237988 | ENSG00000108679 | ENSG00000111537 |
| NLRC5 | OAS3 | APOL6 | PSME2 | PTGES3P1 | DLL4 |
| ENSG00000140853 | ENSG00000111331 | ENSG00000221963 | ENSG00000100911 | ENSG00000234518 | ENSG00000128917 |
| TRIM69 | ALPK1 | PSMB9 | NFE2L3 | FAS | PLAAT4 |
| ENSG00000185880 | ENSG00000073331 | ENSG00000240065 | ENSG00000050344 | ENSG00000026103 | ENSG00000133321 |
| HELZ2 | BCL2L14 | IFI35 | TRIM21 | UBD | IRF9 |
| ENSG00000130589 | ENSG00000121380 | ENSG00000068079 | ENSG00000132109 | ENSG00000213886 | ENSG00000213928 |
| TNFSF4 | RGS1 | SSTR2 | SMTNL1 | TIFA | TMEM229B |
| ENSG00000117586 | ENSG00000090104 | ENSG00000180616 | ENSG00000214872 | ENSG00000145365 | ENSG00000198133 |
| PSME1 | DDX60 | CASZ1 | HERC6 | CASP17P | SAMD9L |
| ENSG00000092010 | ENSG00000137628 | ENSG00000130940 | ENSG00000138642 | ENSG00000235505 | ENSG00000177409 |
| USP18 | NFIX | PSMB10 | GBP6 | DTX3L | PTPRK |
| ENSG00000184979 | ENSG00000008441 | ENSG00000205220 | ENSG00000183347 | ENSG00000163840 | ENSG00000152894 |
| IFIT3 | KLHDC7B | TAPBP | PARP12 | TAP2 | RCN1 |
| ENSG00000119917 | ENSG00000130487 | ENSG00000231925 | ENSG00000059378 | ENSG00000204267 | ENSG00000049449 |
| GPR174 | TAP1 | PARP10 | GBP3 | HLA-F | IFIH1 |
| ENSG00000147138 | ENSG00000168394 | ENSG00000178685 | ENSG00000117226 | ENSG00000204642 | ENSG00000115267 |
| GPR155 | PARP11 | LINC02328 | ERVK-28 | MX1 | TRIM22 |
| ENSG00000163328 | ENSG00000111224 | ENSG00000258733 | ENSG00000267696 | ENSG00000157601 | ENSG00000132274 |
| IL12RB1 | TRAFD1 | IRF1-AS1 | OASL | ACOT7 | GCH1 |
| ENSG00000096996 | ENSG00000135148 | ENSG00000197536 | ENSG00000135114 | ENSG00000097021 | ENSG00000131979 |
| PARP3 | OPTN | MOV10 | ISG15 | MDGA1 | SAMD4A |
| ENSG00000041880 | ENSG00000123240 | ENSG00000155363 | ENSG00000187608 | ENSG00000112139 | ENSG00000020577 |
| SP110 | IL15 | CEACAM1 | CASP4 | PCGF5 | NMI |
| ENSG00000135899 | ENSG00000164136 | ENSG00000079385 | ENSG00000196954 | ENSG00000180628 | ENSG00000123609 |
| SYNPO2 | IFIT5 | CXCL10 | C4BPB | TRANK1 | GSDMD |
| ENSG00000172403 | ENSG00000152778 | ENSG00000169245 | ENSG00000123843 | ENSG00000168016 | ENSG00000104518 |
| CD69 | IF16 | DHX58 | RAB1AP1 | OAS2 | UBA7 |
| ENSG00000110848 | ENSG00000126709 | ENSG00000108771 | ENSG00000229023 | ENSG00000111335 | ENSG00000182179 |
| BISPR | PSMB8 | APOBEC3F | PLAT | DDX58 | HERC5 |
| ENSG00000282851 | ENSG00000204264 | ENSG00000128394 | ENSG00000104368 | ENSG00000107201 | ENSG00000138646 |
| E2F3P1 | DLG3 | PARP14 | CARD16 | CALHM6 | APOBEC3G |
| ENSG00000267046 | ENSG00000082458 | ENSG00000173193 | ENSG00000204397 | ENSG00000188820 | ENSG00000239713 |
| TOMM20P2 | LAG3 | HLA-A | TAFA2 | IFI27 | MX2 |
| ENSG00000255987 | ENSG00000089692 | ENSG00000206503 | ENSG00000198673 | ENSG00000165949 | ENSG00000183486 |
| BTN3A1 | IFIT1 | USP41 | RHEBL1 | BTN3A3 | TMEM140 |
| ENSG00000026950 | ENSG00000185745 | ENSG00000161133 | ENSG00000167550 | ENSG00000111801 | ENSG00000146859 |
| RNF213 | CCL8 | ISG20 | BST2 | IFITM3 | LINC01949 |
| ENSG00000173821 | ENSG00000108700 | ENSG00000172183 | ENSG00000130303 | ENSG00000142089 | ENSG00000233828 |
| CLEC2B | LAP3 | LY6E | CCDC194 | BATF2 | EIF2AK2 |
| ENSG00000110852 | ENSG00000002549 | ENSG00000160932 | ENSG00000269720 | ENSG00000168062 | ENSG00000055332 |
| TNFSF13B | LINC02528 | DGLUCY | VAMP5 | NUB1 | SAMD9 |
| ENSG00000102524 | ENSG00000226004 | ENSG00000133943 | ENSG00000168899 | ENSG00000013374 | ENSG00000205413 |
| STAT2 | RNF213-AS1 | ZDHHC4P1 | PGAP1 | IFIT2 | OAS1 |
| ENSG00000170581 | ENSG00000263069 | ENSG00000238025 | ENSG00000197121 | ENSG00000119922 | ENSG00000089127 |
| CD38 | XRN1 | GVINP1 | NT5C3A | LRRN2 | FBXO39 |
| ENSG00000004468 | ENSG00000114127 | ENSG00000254838 | ENSG00000122643 | ENSG00000170382 | ENSG00000177294 |

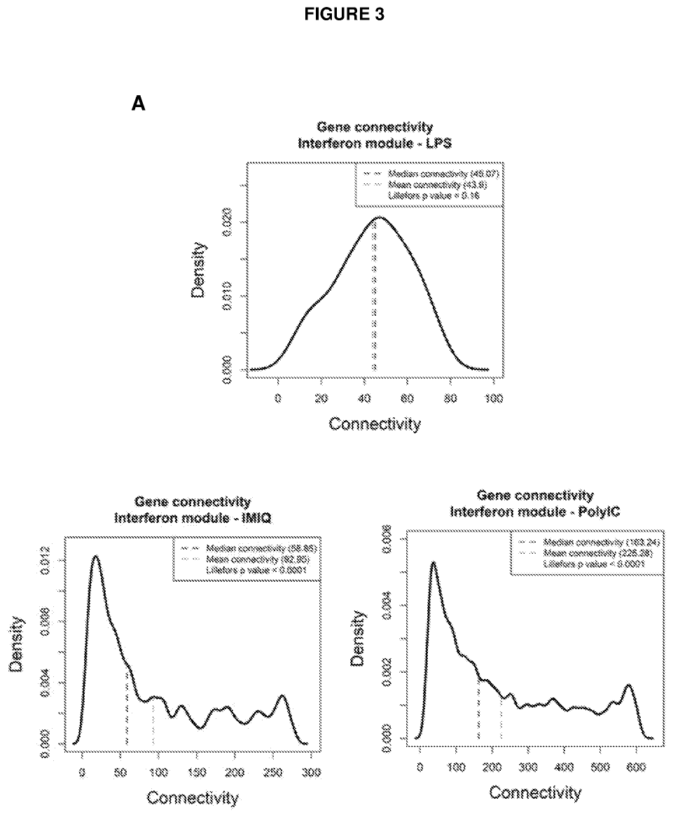

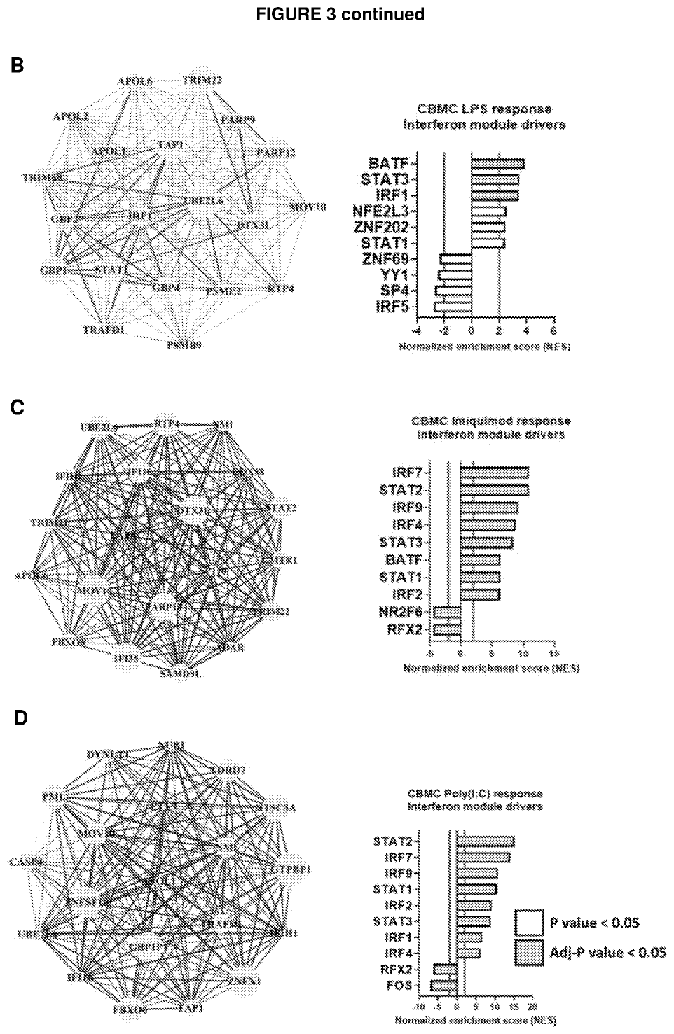

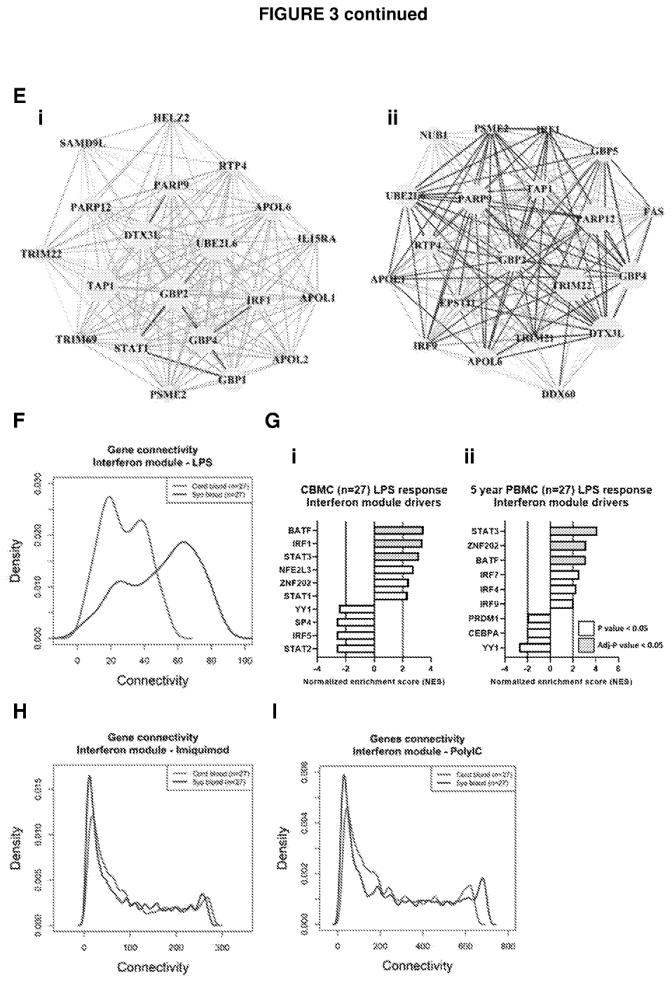

Next, the gene network patterns between the respective responses were compared. First, module preservation statistics were calculated, with the results showing that the LPS-induced IFN module was highly preserved within the IFN modules of the imiquimod and Poly(I:C) responses but not vice versa (data not shown). The IFN modules associated with the imiquimod and Poly(I:C) responses were preserved within one another and the proinflammatory modules were preserved between all responses (data not shown). Second, the ranked gene expression and ranked connectivity was calculated to compare modules. A prominent disparity was observed between expression magnitude (rho=0.88 & 0.82) and intra-module connectivity (rho=0.57 & 0.59) between the cord blood LPS-induced IFN module genes and the same genes following Imiquimod and Poly(I:C) stimulation, respectively (FIG. 2H). To examine connectivity within modules, the connectivity density across all genes in each module was plotted, which identified the most connected genes (FIG. 3A,FIG. 3B-D; left panel). The connectivity of the LPS-induced IFN module was characterised by a normal distribution, whereas the viral stimuli produced left-skewed distributions (FIG. 3A). Key IFN signalling genes (e.g. IRF1, STAT1) were present among the most connected genes within the LPS-induced IFN module, however the strength of the most connected genes was reduced compared to the IFN modules of the viral stimuli (FIG. 3B-D; left panel). The LPS-induced proinflammatory module displayed greater connectivity compared to the imiquimod- or Poly(I:C)-induced proinflammatory modules (data not shown). Genes encoding innate immune/proinflammatory cytokines (e.g. IL1A/B, CXCL2/3/8) were among to most connected genes in the proinflammatory modules of all responses at birth (data not shown). In summary, although viral and bacterial stimuli activate overlapping sets of proinflammatory and IFN responses genes, the underlying network structure was markedly different.

### Example 7—Identification of Master Regulators of Innate Immune Function at Birth and Age 5

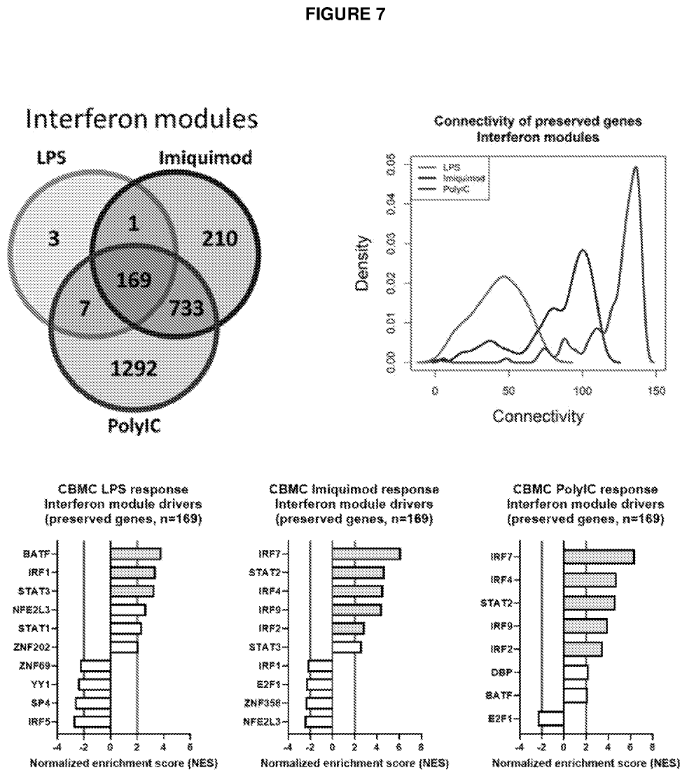

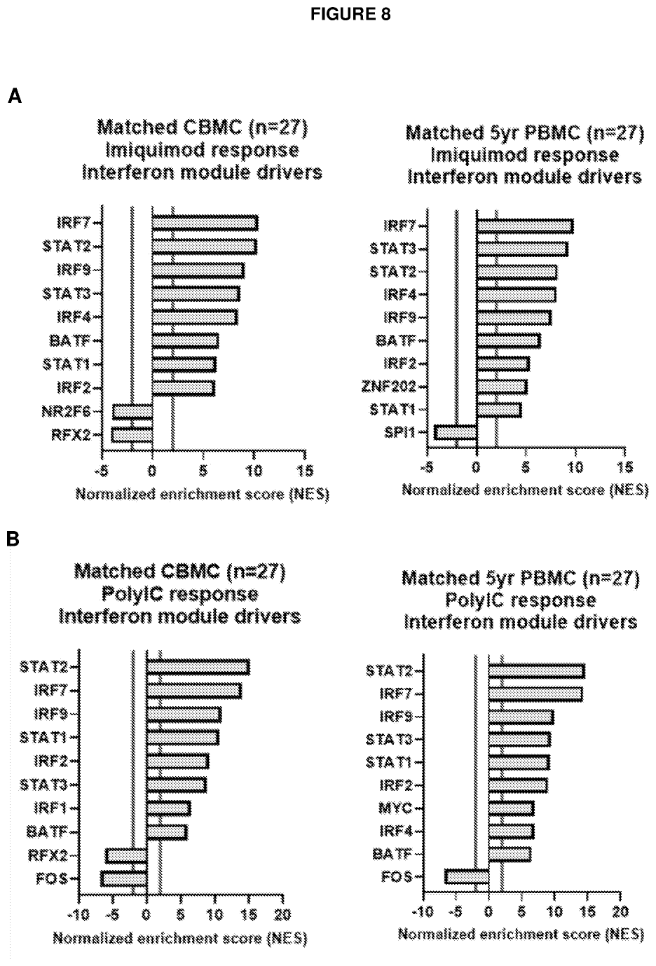

VIPER (Alvarez et al., Nat Genet, 2016, 48:838-847) analysis was employed to identify master regulators which are predicted to drive module connectivity patterns. This approach revealed that the LPS-induced IFN module was putatively driven by BATF3, STAT3 and IRF1 transcription factors (TFs) at birth, whereas the imiquimod- and Poly(I:C)-induced IFN module top drivers included multiple STAT (e.g. STAT2) and IRF (e.g. IRF7) TFs (FIG. 3B-D; right panel, FIG. 6B). IRF1 was found to regulate 52 genes of the 180 LPS-induced IFN module genes (Table 4). The proinflammatory modules for all three responses were enriched for CEBPB, AP-1 (e.g. JUN, FOSL1) and NF-kB (e.g. NFKB2, RELB) (data not shown). Importantly, repeat analyses with input genes restricted to only those preserved from the LPS responses IFN (169/180, 93.89%) (FIG. 7) and proinflammatory (443/2297, 19.29%) modules (data not shown) did not change the data. Finally, gene network patterns between CMBC and matched PBMCs samples (n=27) collected at 5 years was compared. The connectivity of the LPS-induced IFN module was markedly higher at 5 years compared to birth, suggesting that the wiring of this module is subject to developmental regulation (FIGS. 3E & 3F). Additionally, IRF1 enrichment was only identified from cord blood (FIG. 3G). In contrast, the IFN responses provoked by imiquimod and Poly(I:C) stimulation displayed comparatively similar connectivity patterns between birth and age 5 years and, supporting this, the putative drivers were also comparable between birth and 5 years (e.g. STAT2, IRF7) (FIGS. 3H&I, FIG. 8). Imiquimod and poly(I:C) proinflammatory modules were characterised by reduced intra-module connectivity in blood collected at 5 years compared to birth (data not shown).

TABLE 4
LPS-induced IFN module genes regulated by the IRF1 regulon. Gene symbol
and ENSEMBL accession numbers are provided.

| | | | |
|---|---|---|---|
| CLEC2B | CXCL9 | ERVK-28 | FAS |
| ENSG00000110852 | ENSG00000138755 | ENSG00000267696 | ENSG00000026103 |
| FBXO6 | GBP1 | GBP1P1 | GBP2 |
| ENSG00000116663 | ENSG00000117228 | ENSG00000225492 | ENSG00000162645 |
| GBP3 | GBP4 | GBP5 | GBP6 |
| ENSG00000117226 | ENSG00000162654 | ENSG00000154451 | ENSG00000183347 |
| GPR174 | HAPLN3 | IDO1 | IFNG |
| ENSG00000147138 | ENSG00000140511 | ENSG00000131203 | ENSG00000111537 |
| IL12RB1 | IL15 | IL 15RA | LINC02328 |
| ENSG00000096996 | ENSG00000164136 | ENSG00000134470 | ENSG00000258733 |
| NFIX | NLRC5 | OR211P | PARP3 |
| ENSG00000008441 | ENSG00000140853 | ENSG00000237988 | ENSG00000041880 |
| PLAAT4 | PSMB10 | PSMB9 | PSME1 |
| ENSG00000133321 | ENSG00000205220 | ENSG00000240065 | ENSG00000092010 |
| PSME2 | PTGES3P1 | SAMD4A | SOCS1 |
| ENSG00000100911 | ENSG00000234518 | ENSG00000020577 | ENSG00000185338 |
| STAT1 | SYNPO2 | TAP1 | TAPBP |
| ENSG00000115415 | ENSG00000172403 | ENSG00000168394 | ENSG00000231925 |
| TCAF2 | TCAF2C | TCAF2P1 | TIFA |
| ENSG00000170379 | ENSG00000283528 | ENSG00000159860 | ENSG00000145365 |
| TRAFD1 | UBD | UBE2L6 | USP30-AS1 |
| ENSG00000135148 | ENSG00000213886 | ENSG00000156587 | ENSG00000256262 |

### Example 8—Innate Immune Responses at Birth Predict sLRI in the First Year of Life

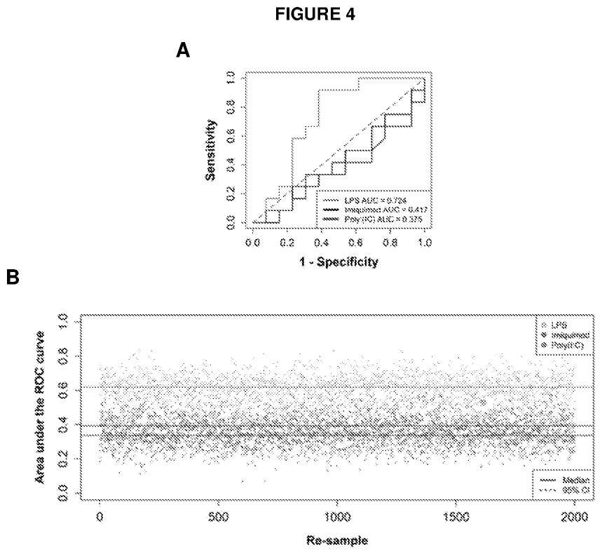

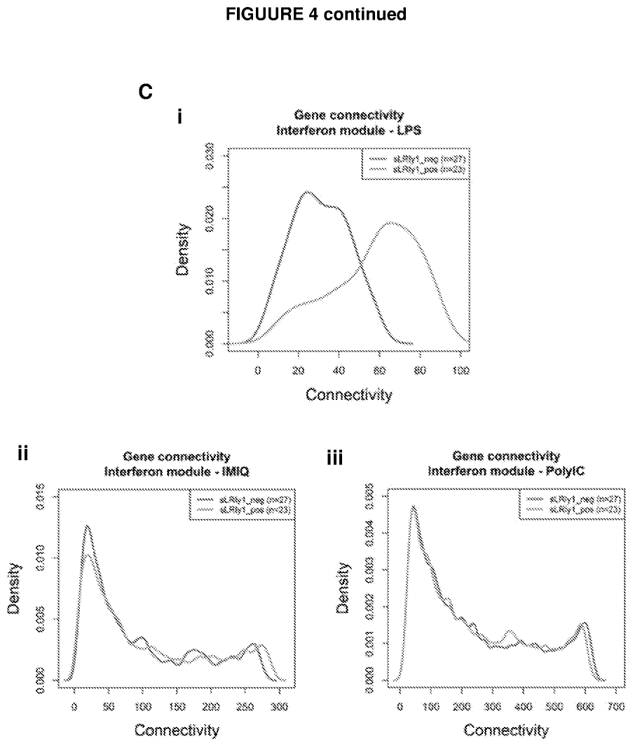

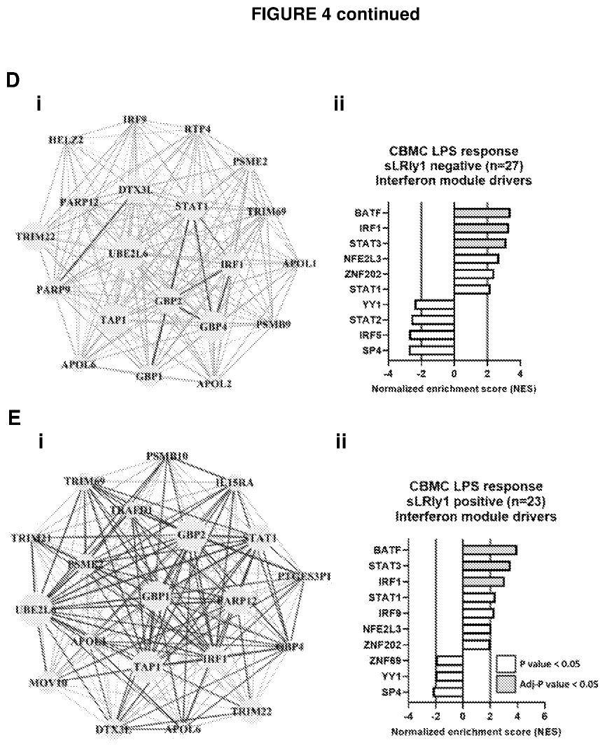

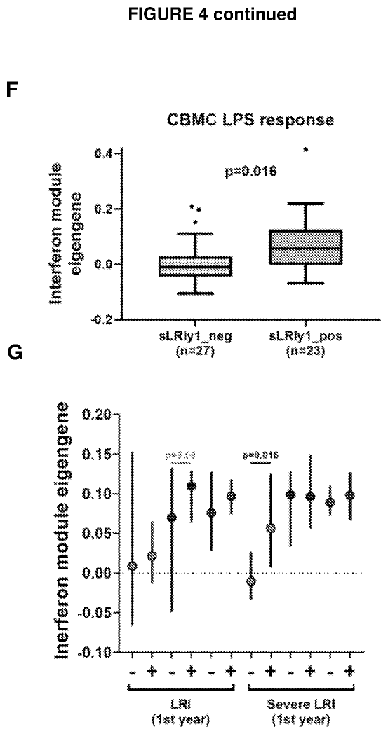

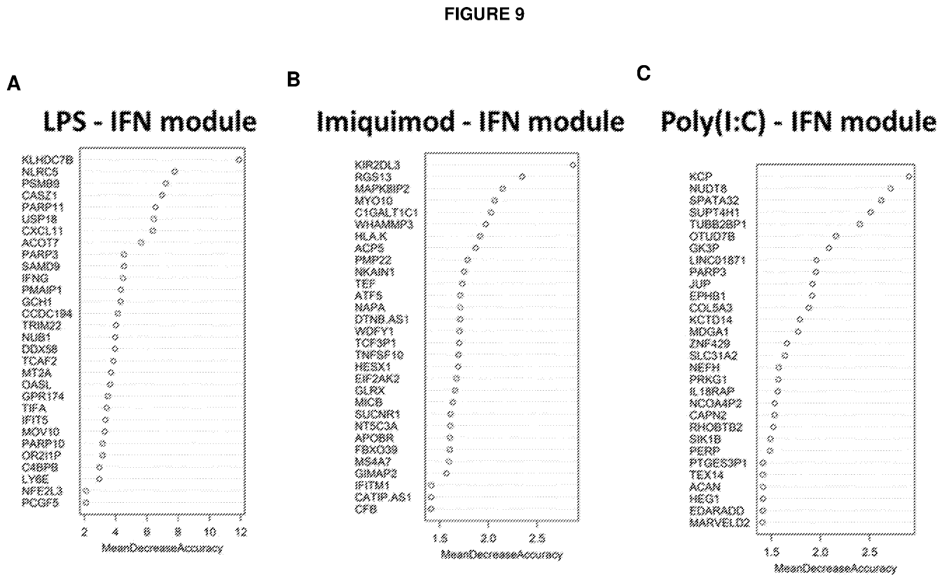

To determine if innate immune responses at birth could predict sLRIs in the first year of life, the data set was randomly assigned into training (50%, n=25) and validation sets (50%, n=25) and a random forest classifier was trained on the CBMC IFN modules. Strikingly, the classifier trained on the LPS-induced IFN module genes could predict sLRIs in the first year of life with an accuracy of 72% in the validation data set (Area under the ROC curve=0.724) (FIG. 4A, FIG. 9). In contrast, classifiers built from the Imiquimod or Poly(I:C) data were not predictive of sLRIs in the first year of life (FIG. 4A). The predictive random forest model also showed that some of the 180 genes provided more accuracy than others, which were quantified by mean decrease Gini or mean decrease accuracy (Table 5 and FIG. 9A). For Gini Importance scoring, a statistical threshold was used (2 median absolute deviations above the median) to determine which genes from the model were the most predictive. 14 genes were above this threshold (Table 5).

TABLE 5
Top filtered genes scored by Gini Importance from the
LPS-induced IFN module.

| Gene Symbol | ENSEMBL ID | MeanDecreaseGini |
|---|---|---|
| KLHDC7B | ENSG00000130487 | 0.398792717 |
| IFNG | ENSG00000111537 | 0.248785492 |
| CASZ1 | ENSG00000130940 | 0.217869997 |
| PSMB9 | ENSG00000240065 | 0.206963824 |
| PARP3 | ENSG00000041880 | 0.188704302 |
| ACOT7 | ENSG00000097021 | 0.187326939 |
| NUB1 | ENSG00000013374 | 0.182121626 |
| USP18 | ENSG00000184979 | 0.179460628 |
| NLRC5 | ENSG00000140853 | 0.173439287 |
| CCDC194 | ENSG00000269720 | 0.168838173 |
| GCH1 | ENSG00000131979 | 0.164823734 |
| PARP11 | ENSG00000111224 | 0.162908602 |
| CXCL11 | ENSG00000169248 | 0.1562304 |
| PMAIP1 | ENSG00000141682 | 0.156106684 |

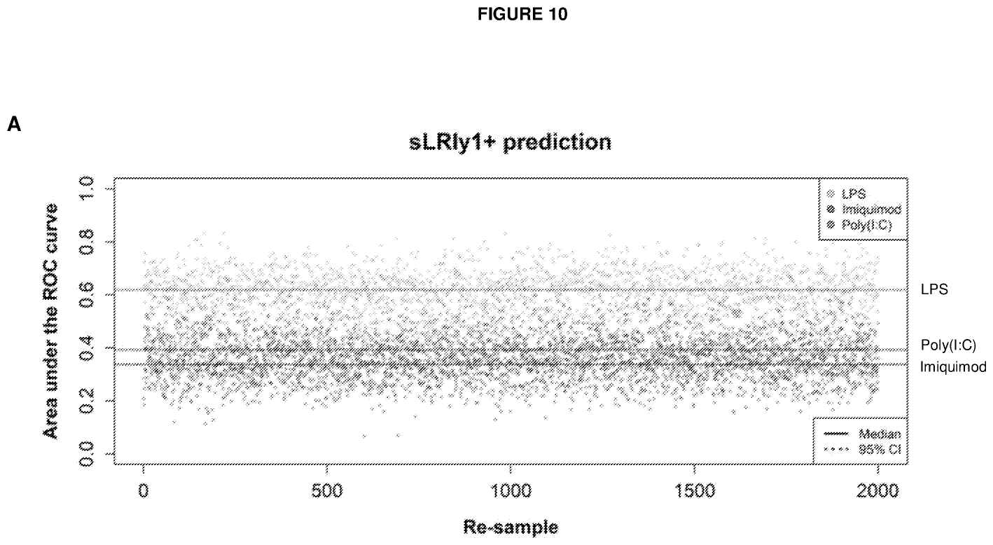

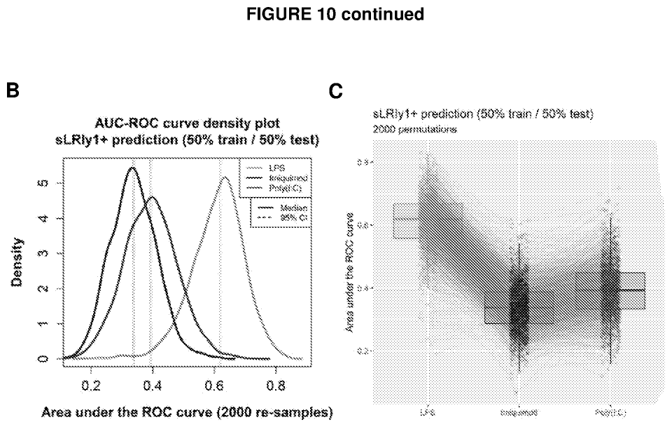

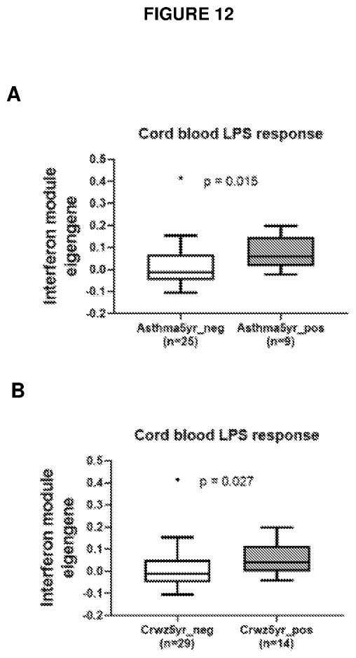

To test whether this finding was reproducible given the relatively small sample numbers available as input, the analysis was repeated by randomly re-sampling subject membership in the training/validation sets, and again it was found that only the LPS-induced IFN module genes could predict sLRIs in infancy better than chance on average (FIG. 10). Further, markedly different connectivity patterns were observed for the LPS-induced IFN module with respect to sLRI susceptibility in infancy, and this was not evident from the imiquimod or Poly(I:C) IFN modules (FIGS. 4B&C). Specifically, susceptible individuals had stronger gene network patterns for the LPS-induced IFN module, although the putative drivers of the response were comparable (IRF1, STAT3, BATF) (FIG. 4B,D-E (i)). Further, restricting the Imiquimod and Poly(I:C) responses to only those genes of the LPS-induced IFN module did not exhibit noticeable differences in connectivity patterns or drivers in relation to sLRI susceptibility in infancy FIG. 11). Whist the connectivity density plot of the LPS-induced IFN module of CBMCs of susceptible individuals (FIG. 4B) resembled the overall connectivity density of the 5 year PBMC connectivity (FIG. 3F), the intra-module connectivity was not related (data not shown), suggesting the similarity emerges from different processes. Module eigengenes were also calculated to summarise overall module expression and correlate with clinical traits. The data showed that the cord blood LPS-induced IFN module eigengene stratified individuals susceptible to sLRIs in the first year of life (p=0.016), as well as those with asthma (p=0.015) and current wheeze (p=0.02) at 5 years of age (FIG. 4F, FIG. 12). This result was only significant for the LPS responses, and was specific for the IFN module (FIG. 4G, FIG. 12).

### Example 9—Validation of Interferon Responses at Birth in External Cohorts

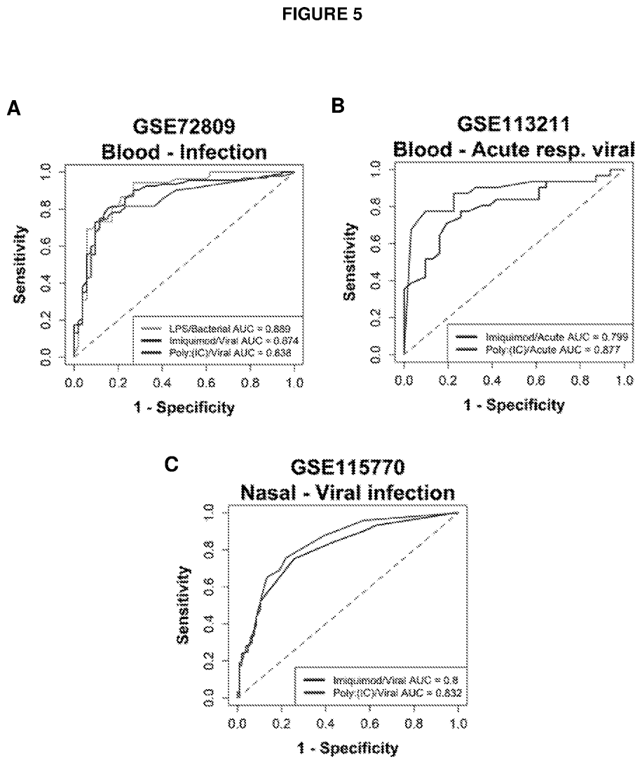

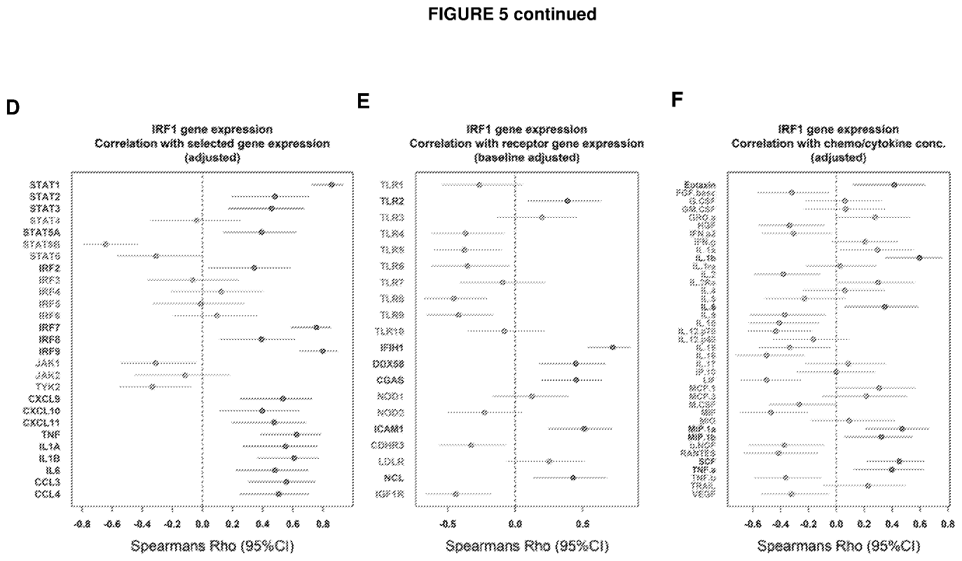

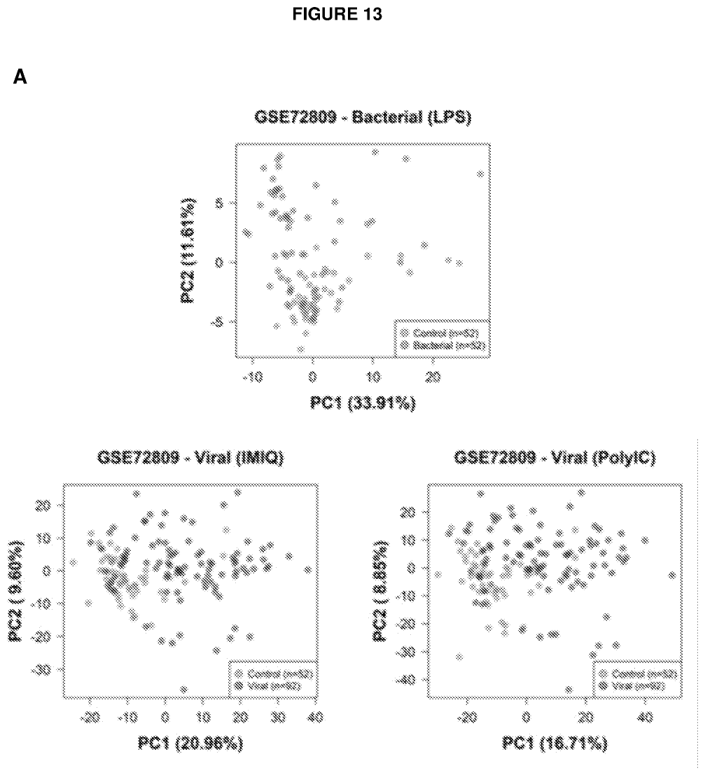

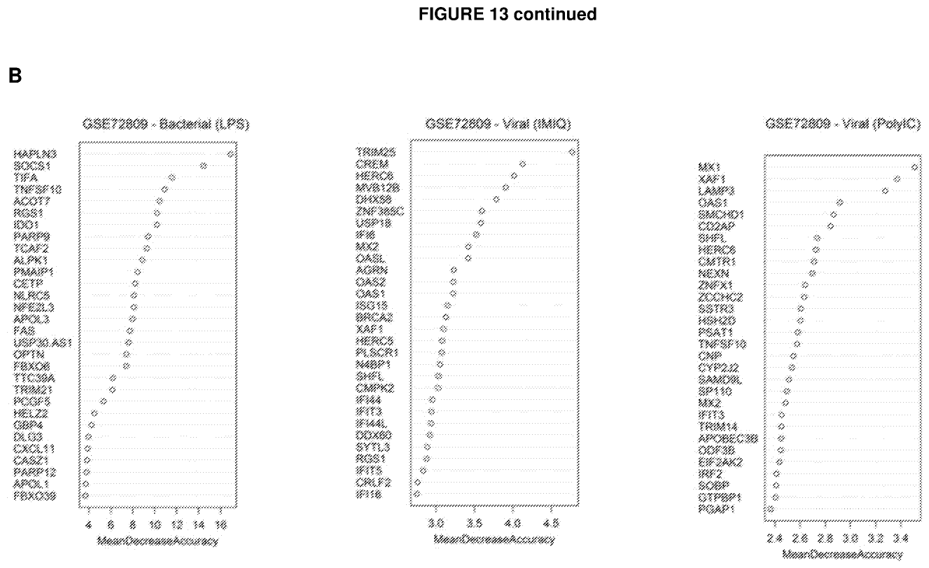

To investigate whether the above findings related to IFN module gene expression profiles induced by CBMC in culture are reflective of naturally occurring IFN responses to childhood infection in vivo, RF classifiers were trained on the CBMC data, and used to classify samples derived from a series of publicly available data sets from the Gene Expression Omnibus. The first data set comprised whole blood gene expression profiles from children (<17 yrs) with febrile illnesses requiring hospitalisation with confirmed bacterial (n=52) or viral (n=92) infections versus healthy controls (n=52) (GSE72809; Herberg et al., JAMA, 2016, 316:835-845). RF classifiers trained on the LPS and Imiquimod/Poly(I:C) data were found to accurately predict children with bacterial (AUC=0.889) and viral (AUC=0.874/0.838) infections, respectively (FIG. 5A-C, FIG. 13). The second data set consisted of PBMC samples from infants (<18 mo, n=30) and young children (18 mo-5 yrs, n=32) who were hospitalized with acute viral bronchiolitis (GSE113211; Jones et al., Am J Respir Crit Care Med, 2019, 199:1537-1549). Classifiers built on unstimulated and either Imiquimod (AUC=0.8) or Poly(I:C) (AUC=0.877) treated CBMCs could accurately stratify samples collected during the acute illness compared to matched post-convalescent samples (symptom-free, 8.8±2.5 weeks post-infection), independent of age. The models performed well for infants (AUC=0.922, Poly(I:C); AUC=0.827, Imiquimod) and children (AUC=0.789, Poly(I:C); AUC=0.842, Imiquimod) separately (data not shown). The third data set consisted of nasal-derived gene expression profiles from study visits of asthmatic children (6-17 yrs) with viral-related or non-viral “cold”-like illness (1-6 days post-onset), some of which later experienced exacerbations (n=83, 58 were viral-positive) (GSE115770; Altman et al., Nat Immunol, 2019, 20:637-651). Symptomatic children with respiratory viral infections were accurately predicted from symptomatic, yet virus-negative, children from Imiquimod (AUC=0.8) and Poly(I:C) (AUC=0.832) defined RF classifiers (FIG. 5E). Additionally, there was comparable accuracy classifying virus-positive and virus-negative asthmatic children who subsequently experienced an exacerbation (within 10 days of symptom onset) (data not shown). In the same study, peripheral blood-derived gene expression profiles of viral versus non-viral exacerbations were classified more accurately from the Imiquimod (AUC=0.671) than the Poly(I:C) defined classifier (AUC=0.627) (data not shown). However, performance was poor when predicting viral positive versus viral negative asthmatics independent of exacerbations from blood expression profiles in this cohort (data not shown).

### Example 10—Multi-Omic Integration

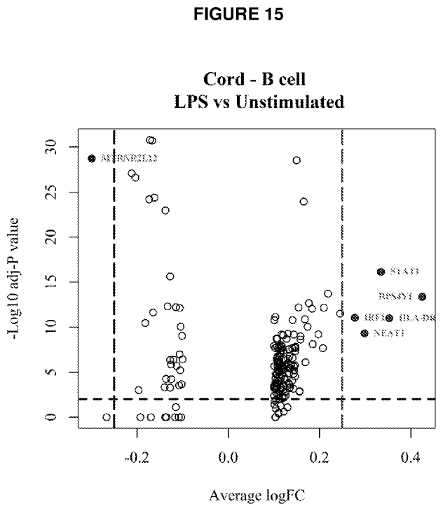

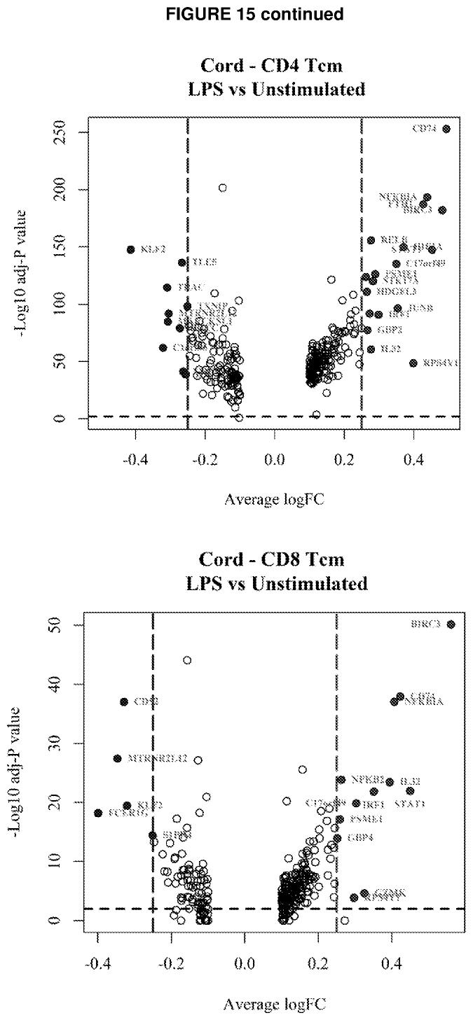

Lastly, multi-omic data integration (DIABLO; Singh et al., Bioinformatics, 2019, 35:3055-3062) was employed to identify correlated molecular features across biological layers which confer sLRI risk. Input data consisted of CBMC baseline immune cell type proportions (n=8), variable mRNA transcripts (n=6353), VIPER-derived regulon activity scores (n=1224), and cytokine/chemokine proteins (n=39). The data reinforced that LPS-induced IFN-signalling transcripts (IRF9, STAT1, GBP2/4) and IRF1 activity were key determinants of risk for sLRI in the first year of life, in combination with T cell, monocyte and DC cell types, immune (HOXB4, NFIX) regulators, and proinflammatory cytokines/chemokines (IL-1β, MIP1α, MIF) (Data not shown). B cells and T cells collected from cord blood were also shown to be the source of the IFN signal as indicated by upregulation of IRF1 and STAT1 when stimulated by LPS (FIG. 15). The LPS-stimulated monocyte/dendritic cell and NK cells didn't upregulate these genes (data not shown).

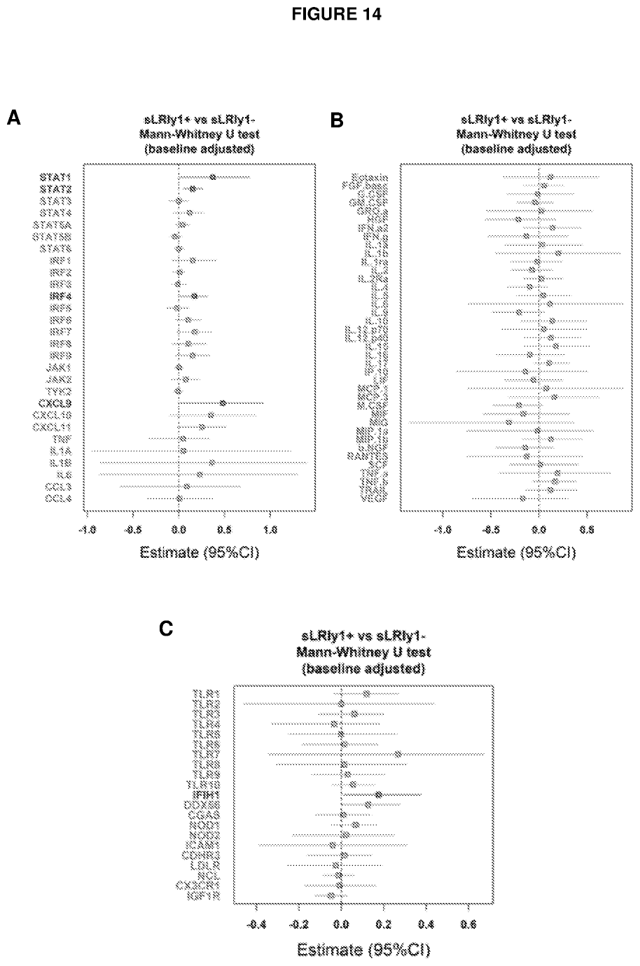

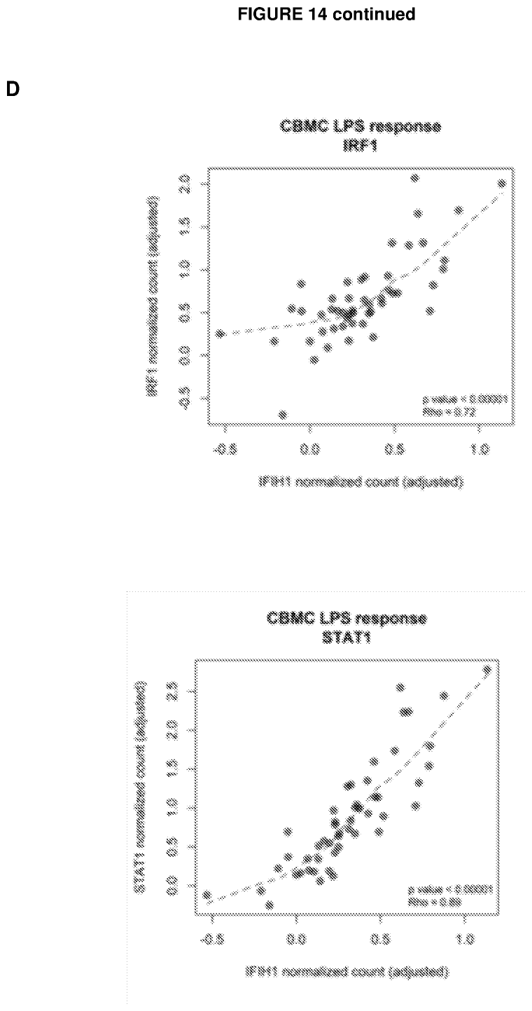

As the LPS-induced IRF1 activity was separately identified from network, master regulator, and integrative analyses, we further investigated IRF1 gene expression correlations. IRF1 gene expression at birth positively correlated with selective STAT and IRF family transcription factors (e.g. STAT1, IRF9), proinflammatory mediators (e.g. IL-1β, IL-6, CCL3/MIP-1α), and viral-related receptors (e.g. ICAM1, IFIH1) (FIG. 5D-F). Additionally, CBMC STAT1 and IFIH1 gene expression was higher in response to LPS among individuals who were susceptible to sLRIs in infancy, and IFIH1 expression correlated with IRF1 and STAT1 expression (FIG. 14).

In summary, severe viral lower respiratory tract infections (sLRIs) are a leading cause of hospitalization in infants and children and constitute a major risk factor for subsequent asthma development. Whilst it is increasingly recognised that bacterial and viral pathogens interact to drive the pathogenesis of sLRIs, the underlying innate immune mechanisms are not well understood. To address this knowledge gap, a multi-omic approach was employed as described in the Examples herein to systematically profile innate immune responses to bacterial (LPS/TLR4) and viral (Poly(I:C)/TLR3; Imiquimod/TLR7) stimuli at birth and investigated response patterns associated with susceptibility to sLRI in the first year of life. The data showed that whilst innate immune responses to the panel of stimuli comprised overlapping proinflammatory and IFN-mediated gene expression programs, the LPS but not Poly(I:C)/Imiquimod response profiles were predictive of sLRI. Moreover, susceptibility was determined by activation of a network of IFN genes, and the connectivity patterns of this network in cord blood LPS responses were strikingly exaggerated among infants at risk of sLRI. Furthermore, the connectivity pattern of these genes was also highly variable between the cord and 5 yr LPS responses, suggesting that the same mechanisms that determine sLRI risk are subject to developmental regulation. These findings were specific for the LPS induced IFN responses and were not observed for IFN responses provoked by TLR3/7 stimulation, nor from proinflammatory genes of any response tested, suggesting that the wiring of the LPS response is specifically altered in children who are at heightened risk for sLRI in infancy. It is noteworthy that expression of the LPS-induced IFN module was not associated with mild (non-wheezy/non-febrile) lower respiratory tract infections, highlighting the relationship of these findings specifically to infection severity. Master regulator analysis identified IRF1 as a key driver of LPS-induced IFN responses at birth. By age 5, the putative activity of IRF1 was replaced by other members of the IRF transcription factor family, including IRF7. In contrast, IRF7 was the dominant driver of TLR3/7 IFN response at birth and 5 years. Finally, employing DIABLO we identified a multi-omic signature associated with sLRI risk, which featured IRF1 regulation and IFN genes in association with proinflammatory cytokines and immune regulators. In summary, our findings suggest that susceptibility to sLRI in the first year of life is primarily determined by anti-bacterial versus anti-viral innate immune pathways, provides a rationale for identification of at-risk infants for early intervention, and identifies targets for drug development.

Recurrent episodes of Rhinovirus-induced wheezing in infancy are often the first indication of early-onset asthma, as demonstrated from prospective cohort studies including CAS (Kusel et al., J Allergy Clin Immunol, 2007, 119:1105-1110) and COAST (Jackson et al., Am J Respir Crit Care Med, 2008, 178:667-672). However, viral infections are routinely detected in asthmatic children in the absence of severe symptoms and recurrent sLRIs across early life alone is not sufficient to drive asthma development, suggesting involvement of additional disease co-factors. In this regard, the data presented herein suggests that responses to pathogenic bacteria are more important determinants of sLRI susceptibility than responses to viral stimuli. Notably, it has been previously shown that over the first 5 years of life, the shift towards increase abundance of these bacterial communities in the airway microbiome frequently precedes viral detection or the onset of respiratory symptoms. This suggests that the presence of pathogenic bacteria may “prime” the airways for expression of severe symptoms during subsequent viral infections.

The present study has found that heightened LPS-induced IFN responses/gene network connectivity patterns at birth conferred risk of viral sLRIs in infancy, which is surprising given that IFN responses are almost universally protective during acute viral infections. In the present study, systems biology was used to identify IRF1 as a master regulator of the LPS-induced IFN network. IRF1 promotes the constitutive expression of interferon-mediated antiviral programs at baseline and the inducible expression of these programs triggered by respiratory viral infections. However, IRF1 is not essential for the induction of interferon programs and provides a complementary rather than essential role in antiviral immunity. The Examples as described herein show that LPS-induced IRF1 gene expression at birth is associated with distinct IFN-signalling mediators (e.g. STAT1 but not JAK1/TYK2) as well as proinflammatory gene/cytokine expression (e.g. CXCL9/10/11, IL1B) (FIG. 5E-G). This is consistent with evidence that Type I IFN-activated IRF1 promotes the induction of specific pro-inflammatory genes. Further, IRF1 association with viral sensing and attachment related receptors (FIG. 5F) supports a role for IRF1 control of these receptors and may, in part, explain how increased IRF1 activation could prime the innate immune system for exaggerated responses to rhinovirus infections.

Innate immune responses are governed by the coordinated activity of multiple cell types across multiple layers of molecular regulation. For this reason, an integrated biomarker profile of co-expressed features was generated to capture the aggregate response associated with sLRI risk in infancy. Strikingly, IFN-signalling genes were prominent among transcripts of the multi-omic risk profile selected following LPS stimulation of cord blood, aligning with the key finding from the network-based approach (IRF1, STAT1). IFIH1, which encodes the important viral recognition receptor MDA5, was also identified. Further, IFIH1 expression was significantly elevated in susceptible individuals and correlated with IRF1 and STAT1, suggesting that at-risk individuals may dysregulate key viral sensing receptors upon TLR4 activation. Other important IRF1-dependent innate pathways are apparent in the data, including MHC class I regulation (NLRC5, RFX5) which is important for CD8+ T cell activation by DC cross-presentation. Among selected cytokines, IL-1 was particularly strongly connected to IFN related transcript, emphasising linked antiviral and proinflammatory response programs downstream of TLR4 activation Finally, many features selected in the integrated profile of sLRI risk in infancy are directly related to asthma. These include transcripts of asthma risk genes (IRF1, P2RY14, ABO) and features involved in remodelling (MMP7), airway inflammation (NLRC5), Th2-dysregulation (LGALS3BP), as well as cell types (T cells, Monocyte/DCs, pDCs, and cytokines (e.g. IL-1β, IL-16, MIF). The supervised data integration approach as used herein, extends the risk profile across biological layers. This highlights the unique ability of integrative multi-omics methods to extract meaningful information from multiple biological level.

The study focused on CBMC innate immune responses in a single birth cohort, and given than the immune system of newborns is subject to drastic developmental changes in the first weeks and months of life, it is valid to question the extent to which CBMC responses reflect immune responses to infections occurring at later ages during childhood. To address this issue, a random forest classifiers was trained on provided in vitro data and applied to infection-associated host response data derived from external cohorts. It was found that IFN responses induced following LPS or imiquimod/Poly(I:C) stimulation could be used to accurately stratify children presenting to hospital with current bacterial and viral febrile infections, respectively, from whole blood samples. These data argue for the relevance of the in vitro models described here to respiratory infections in vivo. Moreover, the CBMC responses induced by imiquimod or Poly(I:C) were also predictive of respiratory viral infections in infants and children with viral bronchiolitis and asthma exacerbation in the blood and airways, suggesting that the signatures are robust to some extent to variations in cellular composition between circulating blood airway tissue. It was also found that the accuracy of the random forest models was higher when predicting infants (<18 mo) compared to younger (18 mo-5 yrs) (GSE113211; Jones et al., AM J Respir Crit Care, 2019, 199:1537-1549) or older (6-17 yrs) children (GSE115770; Altman et al., Nat Immunol, 2019, 20:637-651). Taken together, this supports that the IFN gene networks identified from our in vitro investigation of cord blood are bona fide response mediators of infection in real world contexts.

In summary, the findings described herein demonstrate that LPS-induced IFN responses at birth predicts risk of sLRI in the first year of life, and identifies cellular and molecular targets that have potential utility in modifying the innate immune trajectory towards sLRI susceptibility and childhood asthma development.

1. A method for determining whether an individual has increased susceptibility to respiratory infections, the method comprising:
contacting a sample comprising cord blood mononuclear cells (CBMCs) from the individual with a TLR4 agonist;
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs;
wherein differential expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs contacted with the TLR4 agonist compared to CBMCs not contacted with the TLR4 agonist indicates the individual is at increased susceptibility to respiratory infections.

2. A method for determining whether an individual has increased susceptibility to respiratory infections, the method comprising:
contacting a sample comprising cord blood mononuclear cells (CBMCs) from the individual with a TLR4 agonist;
measuring levels of expression of interferon module biomarkers in the CBMCs;
wherein differential expression of interferon module biomarkers in the CBMCs contacted with the TLR4 agonist compared to CBMCs not contacted with the TLR4 agonist indicates the individual is at increased susceptibility to respiratory infections.

3. A method for determining whether an individual has increased susceptibility to respiratory infections, the method comprising:
contacting a sample comprising cord blood mononuclear cells (CBMCs) from the individual with a TLR4 agonist;
measuring levels of expression of IFN module biomarkers regulated by the IRF1 regulon in the CBMCs;
wherein differential expression of IFN module biomarkers regulated by the IRF1 regulon in the CBMCs contacted with the TLR4 agonist compared to CBMCs not contacted with the TLR4 agonist indicates the individual is at increased susceptibility to respiratory infections.

4. A method for determining whether an individual has increased susceptibility to respiratory infections, the method comprising:
contacting a sample comprising cord blood mononuclear cells (CBMCs) from the individual with a TLR4 agonist;
measuring level of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of the biomarkers KLHDC7B, IFNG, CASZ1, PSMB9, PARP3, ACOT7, NUB1, USP18, NLRC5, CCDC194, GCH1, PARP11, CXCL11 and PMAIP1 in the individual compared to a reference data set indicates the individual is at increased susceptibility to respiratory infections.

5. A method for determining whether an individual has increased susceptibility to respiratory infections, the method comprising:
contacting a sample comprising cord blood mononuclear cells (CBMCs) from the individual with a TLR4 agonist;
measuring level of expression of interferon module biomarkers in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of interferon module biomarkers in the individual compared to a reference data set indicates the individual is at increased susceptibility to respiratory infections.

6. A method for determining whether an individual has increased susceptibility to respiratory infections, the method comprising:
contacting a sample comprising cord blood mononuclear cells (CBMCs) from the individual with a TLR4 agonist;
measuring level of expression of IFN module biomarkers regulated by the IRF1 regulon in the CBMCs;
comparing the level of expression of the biomarkers from the individual to a reference data set, wherein the reference data set comprises information on the levels of expression of the same biomarkers in CMBCs contacted with a TLR4 agonist from one of more individual individuals with or without an increased susceptibility to respiratory infections;
wherein the levels of expression of IFN module biomarkers regulated by the IRF1 regulon in the individual compared to a reference data set indicates the individual is at increased susceptibility to respiratory infections.

7. The method according to any one of claims 2 or 5, wherein the interferon module biomarkers are those listed in Table 3.

8. The method according to claim 3 or 6, wherein the IFN module biomarkers regulated by the IRF1 regulon are those listed in Table 4.

9. The method according to any of the preceding claims, wherein the method further comprises a step of applying a machine learning algorithm to the differential or absolute expression of biomarkers thereby indicating the individual is at increased of susceptibility to respiratory infections.

10. The method according to claim 9, wherein the machine learning algorithm is a random forest analysis.

11. The method according to any one of claims 1 to 3, wherein the level of expression is differential expression and the differential expression is a greater than 1.5-fold increase or decrease.

12. The method according to any one of claims 4 to 6, wherein the increased susceptibility is an relative risk or odds ratio of at least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.30, at least 1.35, at least 1.40, at least 1.45, at least 1.50, at least 1.55, at least 1.60, at least 1.65, at least 1.70, at least 1.75, or at least 1.80 compared to the reference data set.

13. The method according to any of the preceding claims, wherein the individual is equal to or less than 1 year old.

14. The method according to any one of the preceding claims, wherein the individual is 1 day, 2 days, 3 days, 4 days, 5 days, 6 days, 7 days, 2 weeks, 1 month, 3 months, 6 months or 1 year old.

15. The method according to any of the preceding claims, wherein the method indicates the individual is at increased susceptibility to respiratory infections when the individual is about 2, about 3, about 4 or about 5 years old; or 2, 3, 4, or 5 years old.

16. The method according to any of the preceding claims, wherein the method indicates the individual is at increased susceptibility to respiratory infections when the individual is at least 5 years old.

17. The method according to any of the preceding claims, wherein the respiratory infections are lower respiratory tract infections.

18. The method according to any of the preceding claims, wherein the respiratory infections are bacterial or viral respiratory infections.

19. The method according to claim 18, wherein the viral respiratory infection is selected from the group consisting of: genus influenza, parainfluenza, coronavirus, adenovirus, metapneumonvirus, rhinovirus and respiratory syncytial virus respiratory infection, preferably the viral respiratory infection is a rhinovirus or respiratory syncytial virus respiratory infection.

20. The method according to claim 18, wherein the bacterial respiratory infection is a Haemophilus influenzae, Staphylococcus aureus or Moraxella spp. respiratory infection.

21. The method according to any of the preceding claims, wherein the respiratory infections are severe lower respiratory tract infections.

22. The method according to any one of the preceding claims, wherein the biomarkers with a differential increase are those listed in Tables 3 and 4, other than IF16, IF127, RHEBL1, CCDC194, BATF2, CARD16, IFIT1, ISG20, IFITM3, VAMP5, TNFSF13B, SAMD9, RNF213-AS1, IFIT2, XRN1, CD38, LRRN2 and CCDC194.

23. The method according to any of the preceding claims, wherein the CBMCs are, or have been, contacted with the TLR4 agonist for at least 4, 6, 12, 18 or 24 hours.

24. The method according to any of the preceding claims, wherein the biomarker is a nucleic acid or amplification product.

25. The method according to any of the preceding claims, wherein the method further comprises administering a treatment to the individual that lowers susceptibility to respiratory infections.

26. The method according to claim 25, wherein the treatment is palivizumab, prednisolone, omalizumab or a polybacterial formulation, or any combinational thereof.

27. The method according to any one of the preceding claims, wherein the sample is cord blood.

28. The method according to any one of claims 1 to 26, wherein the CBMCs are purified from cord blood.

29. The method according to any one of the preceding claims, wherein the CBMCs comprise B cells and T cells, preferably CD4+ and/or CD8+ T cells.

30. The method according to claim 29, wherein the T cells are CD4+ central memory T cells and/or CD8+ central memory T cells.

31. The method according to claim 29 or 30, wherein the level of expression of the biomarkers in B and T cells only are measured in the CBMCs.

32. The method according to any one of claims 29 to 31, wherein the CBMCs further comprise CD14+ monocytes and conventional dendritic cells (cDCs) and optionally plasmacytoid DCs (pDC).

33. The method according to any one of the preceding claims, wherein the TLR4 agonist is selected from the group consisting of lipopolysaccharide (LPS), monophosphoryl lipid A (MPLA), a heat shock protein, S100A8, S100A9, RSV F protein, fibrinogen, heparin sulfate or a fragment thereof, hyaluronic acid or a fragment thereof, nickel, an opoid, α1-acid glycoprotein (AAG), aminoakyl glucoaminide 4-phosphate (AGP), RC-529, murine β-defensin 2, and complete Freund's adjuvant (CFA), preferably the TLR4 agonist is LPS.

34. The method according to claim 33, wherein the TLR4 agonist is derived from a bacterium, optionally the TLR4 agonist is purified or contained in a bacterial preparation.

35. The method according to claim 33 or 34, wherein the TLR4 agonist is LPS and is provided at an effective concentration to stimulate TLR4 activation, preferably the concentration of LPS is between 0.025 ng/ml to 100 ng/ml, more preferably the concentration of LPS is 1 ng/ml.

36. The method according to any one of the preceding claims, wherein the CBMCs are suspended at 1×10⁶ cells/mL before contact with the TLR4 agonist.

37. A kit, panel or microarray comprising diagnostic reagents that bind to or complex individually with each of the biomarkers:
KLHDC7B, IFNG, CASZ1, PSMB9, PARP3, ACOT7, NUB1, USP18, NLRC5, CCDC194, GCH1, PARP11, CXCL11 and PMAIP1;
interferon module biomarkers listed in Table 3; or
IFN module biomarkers regulated by the IRF1 regulon listed in Table 4.

38. A kit, panel or microarray for use or when used according to the method of any one of claims 1 to 28 comprising diagnostic reagents that bind to or complex individually with each of the biomarkers:
KLHDC7B, IFNG, CASZ1, PSMB9, PARP3, ACOT7, NUB1, USP18, NLRC5, CCDC194, GCH1, PARP11, CXCL11 and PMAIP1;
interferon module biomarkers listed in Table 3; or
IFN module biomarkers regulated by the IRF1 regulon listed in Table 4.

39. An assay comprising
contacting B and T cells from cord blood from the individual with a TLR4 agonist; and
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the B and T cells.

40. An assay comprising
contacting B and T cells from cord blood from the individual with a TLR4 agonist; and
measuring levels of expression of interferon module biomarkers in the B and T cells.

41. An assay comprising
contacting B and T cells from cord blood from the individual with a TLR4 agonist; and
measuring levels of expression of IFN module biomarkers regulated by the IRF1 regulon in the B and T cells.

42. An assay comprising
contacting cord blood mononuclear cells (CBMCs) from the individual with a TLR4 agonist; and
measuring levels of expression of biomarkers KLHDC7B, IFNG, CASZ1, PSMB9, PARP3, ACOT7, NUB1, USP18, NLRC5, CCDC194, GCH1, PARP11, CXCL11 and PMAIP1 in the CBMCs.

43. An assay comprising
contacting cord blood mononuclear cells (CBMCs) from the individual with a TLR4 agonist; and
measuring levels of expression of interferon module biomarkers in the CBMCs.

44. An assay comprising
contacting cord blood mononuclear cells (CBMCs) from the individual with a TLR4 agonist; and
measuring levels of expression of IFN module biomarkers regulated by the IRF1 regulon in the CBMCs.
